(19) United States
(12) Patent Application Publication
(10) Pub. No.: US 2011/0020837 A1
(43) Pub. Date: Jan. 27, 2011

(54) Method for isolating or identifying a target protein interacting with a lipid in a cell

(75) Inventors: Per Haberkant, Utrecht (NL); Hendrik Sprong, Soesterberg (NL); Gerardus Franciscus Bernardus Petrus Van Meer, Utrecht (NL)

Correspondence Address: TRASKBRITT, P.C., P.O. BOX 2550, SALT LAKE CITY, UT, 84110, US

(73) Assignee: Universiteit Utrecht Holding B.V., Utrecht (NL)

(21) Appl. No.: 12/803,886

(22) Filed: Jul. 7, 2010

Related U.S. Application Data
(60) Provisional application No. 61/223,392, filed on Jul. 7, 2009.

(51) Int. Cl. G01N 33/53; C12Q 1/02; C07K 1/14

(52) U.S. Cl. 435/7.1; 435/29; 530/412

(57) ABSTRACT

The invention is in the field of molecular biology and cell biology. It provides tools and methods for studying the interaction of proteins and lipids in vivo as well as in vitro. The invention relates to a method for isolating or identifying a target protein interacting with a lipid in a cell. This method employs novel dual-labeled lipid precursors such as fatty acids or their derivatives. These lipid precurors comprise two functional groups: a photoactivatable group, such as a diazirine ring, as well as a terminal alkyne or azide moiety.

### PRIORITY CLAIM

This application claims the benefit of the filing date of U.S. Provisional Patent Application Ser. No. 61/223,392, filed Jul. 7, 2009, the entire disclosure of which is hereby incorporated herein by this reference.

### TECHNICAL FIELD

The invention is in the field of molecular biology and cell biology. It provides tools and methods for studying the interaction of proteins and lipids in vivo as well as in vitro.

### BACKGROUND

The human genome encodes approximately 30,000 genes. Taking into account splice variants and post-translational modifications such as phosphorylations and glycosylations the overall number of protein species is far higher. Interactomics has provided a good overview of protein-protein interactions. However, 30% of the proteins are membrane proteins and many more exert their functions at a membrane surface. The lipidome of eukaryotic cells comprises thousands of different lipids, which raises the need to study the interactions between lipids and proteins on a proteome-wide scale. Radioactive photoactivatable lipids have been used to investigate individual protein-lipid interactions. Some of these probes can be considered as state-of-the-art in terms of minimal perturbance.

### SUMMARY OF THE INVENTION

In order to enrich cross-linked proteins, we developed a method for isolating or identifying a target protein interacting with a lipid comprising the steps of:
(a) providing a lipid precursor having a photoactivatable group and a terminal alkyne or azide group or;
(b) providing two lipid precursors wherein the first comprises a photoactivatable group and the second comprises a terminal alkyne or azide group;
(c) contacting the lipid precursors according to a or b with cells and allowing the precursors to be incorporated into lipids;
(d) exposing the cells to photolysis wherein a target protein interacting with a lipid is covalently attached to the lipid having a terminal alkyne and/or an azide group;
(e) isolating or identifying the target protein by attaching a reporter molecule to the terminal alkyne or azide group.

### BRIEF DESCRIPTION OF THE DRAWINGS

FIG. 1. Panel A, Photoactivatable and clickable fatty acids (pacFAs, 6a, 6b), bifunctional sphingolipids C15pacGlcCer (7), C15pacCer (8) and the reporter molecule biotin-azide (9). The chemical synthesis of pacFAs is described in FIG. 4. Panel B, Strategy for the detection of protein-lipid interactions. Fluo: alexa488.

FIG. 2. In vivo labeling of proteins interacting with lipids. CHO-CGalT cells were labeled with and without 100 μM C15pacFA for 16 hours. Where indicated, cells were UV irradiated. Total membranes were separated from cytosol. The cytosolic proteins were precipitated by CHCl₃/MeOH precipitation and total membranes as well as the cytosolic precipitate were subjected to click reaction conditions using alexa488-azide or biotin-azide. Panel A, Proteins labeled with alexa488-azide were separated by SDS-PAGE and analyzed by in-gel fluorescence. Panel B, Prominent band at 20 kDa is due to an alexa488-azide copper complex (data not shown) Coomassie brilliant blue staining of the gel shown in Panel A. Panel C, Following in vivo photo-affinity labeling, cell lysates were subjected to click reactions with biotin-azide (input) and purified using NeutrAvidin beads (pull down); SN: supernatant. Proteins were separated by SDS-gel, blotted on a PVDF membrane and decorated with an antibody against biotin. Panel D, Proteins eluted from beads were separated by SDS-PAGE and stained with Coomassie. The lanes were cut into ten gel pieces as indicated and analyzed by mass spectrometry as described herein. Prominent band at 15 kDa is due to the monomeric subunits of NeutrAvidin.

FIG. 3. Panel A, Cytosol was incubated with liposomes containing C15pacCer or C15pacGlcCer as indicated. Upon photo-affinity labeling, samples were subjected to click reactions with biotin azide. A representative Western blot is shown (n=4). Panel B, Comparison of biotinylated proteins by Western blotting. Panel C, Samples of B were analyzed by Coomassie staining. Panel D, Western blot analysis of pull down using an anti-biotin antibody.

FIG. 4. Synthesis of photoactivatable and clickable fatty acids (pacFAs). The protected keto fatty acids 4a/b were obtained by coupling trimethylsilyl-protected alkynes 2a/b to methyl 10-chloro-10-oxodecanoate 3 in a Grignard reaction. Simultaneous deprotection yielded the keto-fatty acids 5a/b that were subsequently converted into the photoactivatable fatty acid derivatives as previously described.^{[11,21-23]}

FIG. 5. Panel A, Proof-of-concept for the specific interaction of pacFA with a fatty acid binding protein. An ethanolic solution of C15pacFA was added to a solution of defatted-BSA or lysozyme in PBS as described above. After incubating for 1 hour at room temperature samples were UV-irradiated and 20 μl were subjected to click reaction with biotin-azide. Samples were analyzed by SDS-PAGE, followed by Western blotting and detection via NeutrAvidin coupled to horse radish peroxidase. Panel B, SDS-PAGE and subsequent staining with Coomassie brilliant blue.

FIG. 6. Molecular modeling structures of C15pacFA (Column B), C16pacFA (Column C), C17pacFA (Column D), myristic acid (Column E) and palmitic acid (Column F). Energy minimized structures were generated with ChemBio3D Ultra (version 11.0.1) employing the MM2 force-field. Minimum RMS Gradient was set to 0.1. Lengths were calculated as the distance between the first and the last C-atom.

FIGS. 7A and 7B. Mass spectrometric analysis of photoactivatable and clickable PC species. CHO-CGaIT cells (−5×10⁷ cells) were labeled for 16 hours in delipidated medium in the absence (upper panel) or presence (lower panel) of 100 μM (FIG. 7A) C15pacFA or (FIG. 7B) C16pacFA. Lipids were extracted, dried and subsequently dissolved in 5 mM ammonium acetate in MeOH and subjected to nano-ESI-MS/MS. Shown are PREC184 spectra visualizing choline phosphate-containing lipids. Abbreviations give the total number of carbon atoms of fatty acids attached and the numbers of double bonds within the fatty acid. Transparent bars highlight PC species containing the pacFAs.

FIG. 8. Wavelength scan of C15pacFA in ethanol before (Panel A) and after (Panel B) UV-irradiation. The photoactivatable diazirine ring shows two characteristic maxima at 349.5 nm and at 366.5 nm.

FIG. 9. Panel A, Activation of the photoactivatable diazirine ring was optimized by exposing an ethanolic solution of 10-azi-stearic acid (10-ASA) under the described conditions to UV-light for the indicated time. Absorbance at 349.5 nm was measured to monitor decay of 10-ASA. Panel B, A solution of defatted BSA (1 mg/ml) was irradiated under the same conditions and analyzed by SDS-PAGE. Coomassie staining revealed no degradation of BSA for up to 5 minutes of irradiation.

### DETAILED DESCRIPTION OF THE INVENTION

We developed a method for isolating or identifying a target protein interacting with a lipid in a cell. This method employs novel dual-labeled lipid precursors such as fatty acids or their derivatives. These lipid precursors comprise two functional groups: a photoactivatable group, such as a diazirine ring as well as a terminal alkyne or azide moiety.

The invention therefore relates to a method for isolating or identifying a target protein interacting with a lipid comprising the steps of:
(a) providing a lipid precursor having a photoactivatable group and a terminal alkyne or azide group or;
(b) providing two lipid precursors wherein the first comprises a photoactivatable group and the second comprises a terminal alkyne or azide group;
(c) contacting the lipid precursors according to a or b with cells and allowing the precursors to be incorporated into lipids;
(d) exposing the cells to photolysis wherein a target protein interacting with a lipid is covalently attached to the lipid having a terminal alkyne and/or an azide group;
(e) isolating or identifying the target protein by attaching a reporter molecule to the terminal alkyne or azide group.

The term “lipid precursor” as used herein is meant to relate to any molecule that may be incorporated into a lipid. In particular the term is used to indicate a molecule selected from the group of an acetate, a fatty acid, an acetylCoA, an acylCoA, a ketodihydrosphingosine, a dihydro-sphingosine, a sphingosine, a phytosphingosine, a sphingosine-1-phosphate, a ceramide, a phytoceramide, inositolphosphorylceramide, an inositolphytosphingosine, a glucosylsphingosine, a galactosylsphingosine, a glucosylceramide, a galactosylceramide, sphingomyelin, ceramide phosphorylethanolamine, a cholesterol, a cholesterol ester, a choline, a glycerol-3-phosphate, a diacylglycerol, a phosphatidic acid, tryglyceride, a serine, a threonine, a ethanol amine, biphosphatidylglycerol, dihydroxyacetone phosphate, phosphatidylcholine, phosphatidylethanolamine, phosphatidylcholine, phosphatidylserine, phosphatidylthreonine, a sugar (glucose, galactose, mannose, glucosamine, N-acetylglucosamine, N-acetylgalactosamine, N-acetylneuraminic acid), sialic acid, an inositol, isoprene, mevalonate, farnesyl, geranyl and 2,3-diacylglucosamine 1-phosphate. Fatty acids are preferred precursors because of practical considerations.

The term “photoactivatable group” is used herein to indicate a group capable of becoming covalently bound to another molecule upon irradiation by light, preferably ultraviolet light.

The term “terminal alkyne group” refers to a group with a chemical formula of H—C≡C—R, HC₂R.

In the context of the present invention, R may comprise a lipid precursor or a reporter molecule.

An alkyne group is capable of being covalently linked in a chemical reaction with a molecule containing an azide.

The term “photolysis” is meant to indicate the light induced activation of a photoactivatable group (e.g., diazirine ring) resulting in a reactive species (e.g., carbene) giving a covalent reaction with a molecule in close proximity.

Once the target protein is covalently attached to the lipid, the protein may be identified or visualized by methods known in the art. The process of attaching a reporter molecule to a terminal alkyne or azide group is described in Rostovtsev et al., Angew. Chem. Int. Ed., 2002, and Tornoe et al., J. Org. Chem. 2002.

It was surprisingly found that such lipid precursors were efficiently taken up by living cells and incorporated into lipids so that they can be used to study lipid-protein interactions.

The invention therefore relates to a method as described above wherein the lipid precursors are contacted with cells in vivo.

It was also surprisingly found that the lipid precursors were more efficiently incorporated into some lipids than in others. It was found that the bifunctional fatty acid was incorporated into phosphatidylcholine (PC), phosphatidylserine (PS), phosphatidylethanolamine (PE) species but not into sphingomyelin (SM) species. The invention therefore relates to a method as described above wherein the lipids are selected from the group consisting of phosphatidylcholine (PC), phosphatidylserine (PS) and phosphatidylethanolamine (PE).

A diazirine ring is the smallest existing photoactivatable group. This is advantage because it will only result in minimal structural changes within the lipid precursor. Incorporated into lipids and amino acids it has been successfully used to study protein-lipid as well as protein-protein interactions.^{[1]}

The invention therefore relates to a method as described above wherein the photoactivatable group is a diazirine group.

The terminal alkyne or azide may serve as a non-native and non-perturbing bioorthogonal chemical handle that can be derivatized employing a chemistry that is known as click chemistry.

The term click chemistry as used herein may refer to the copper(I)-catalyzed [3+2]-Huisgen 1,3-dipolar cyclo-addition of terminal alkynes and azides leading to 1,2,3-triazoles. It may also refer to a copper free variant of this reaction that might also be used. (J. M. Baskin, J. A. Prescher, S. T. Laughlin, N. J. Agard, P. V. Chang, I. A. Miller, A. Lo, J. A. Codelli, C. R. Bertozzi, Proc. Natl. Acad. Sci. U.S.A. 2007, 104, 16793.)

Due to the functional groups, the lipid precursors as described herein will be referred to as photoactivatable and clickable lipid precursors and the fatty acids described herein will be referred to as photoactivatable and clickable fatty acids (pacFAs). In case the lipid precursor comprises a terminal alkyne, the reporter molecule may comprise an azide and vice versa.

The invention therefore relates to a method as described above wherein the reporter molecule comprises an alkyne or an azide group.

The technology described herein allows the synthesis of pacFAs with different chain lengths. The position of the photoactivatable group within the fatty acids can be varied depending on the precursors used. Since fatty acids may serve as a building block for the biosynthesis of lipids as well as for their chemical synthesis, pacFAs provide fast access to a great number of photoactivatable and clickable lipids in vivo as well as in vitro.

The approach described here can be divided into two steps: (i) proteins interacting with dually labeled lipids are covalently linked to the lipids upon photolysis, such as UV irradiation. (ii) In a subsequent step, cross-linked products are labeled by a reporter molecule, e.g., alexa488-azide or biotin-azide, by means of click chemistry.

We demonstrated that pacFAs may serve as substrates for cellular metabolism for lipid biosynthesis demonstrating them to mimic their natural counterparts. We herein provide protocols for photo-affinity labeling of proteins interacting with lipids in vitro as well as in vivo. Further we provide protocols for derivatization of photo-affinity-labeled proteins. This enables visualization and purification of cross-linked products.

The advantage over previously reported radioactive photoactivatable lipid precursors is given by a rapid and highly sensitive readout and, even more important, cross-linked products can be purified after respective derivatization by means of click chemistry. Our invention forms the basis for an approach that provides a simple and robust tool to probe for protein-lipid interactions in vivo as well as in vitro.

The lipidome of a eukaryotic cell comprises thousands of lipids. The photoactivatable and clickable lipids recently described by Gubbens et al. (2009) are supposed to identify proteins interacting with the headgroup of one particular lipid. Furthermore, the photoactivatable groups that have been reported have more than ten times the molecular weight of the methyl group they substitute. To which extent these lipids still mimic their natural counterpart is not known.

In contrast, the newly designed fatty acid derivatives described herein serve as precursors for a great number of photoactivatable and clickable lipids. Our data show that these fatty acids are taken up by cells and enter the enzymatic machinery required for the biosynthesis of lipids. This demonstrates that the fatty acids analogues we describe mimic their natural counterparts. Furthermore, they enable in vivo applications.

The present approach provides a simple and robust tool to probe for protein-lipid interactions in vivo as well as in vitro. Bifunctional lipids allow the screening for lipid handling machinery. The possibility to tag a subset of the proteome with a high affinity for lipids opens possibilities for diagnostic applications.

A comparison of protein-lipid interactions under various conditions, such as the presence of inhibitors, temporal knockdowns or during stimulation of signaling pathways, will allow to monitor interactions with defined lipids in vivo and will enable to study the dynamics of these interactions. The identification of key players in the life of individual lipids will help to elucidate their function and will close the gap between proteomics and lipidomics.

### EXAMPLES

### Example 1

### Synthesis of Photoactivatable and Clickable Lipid Precursors

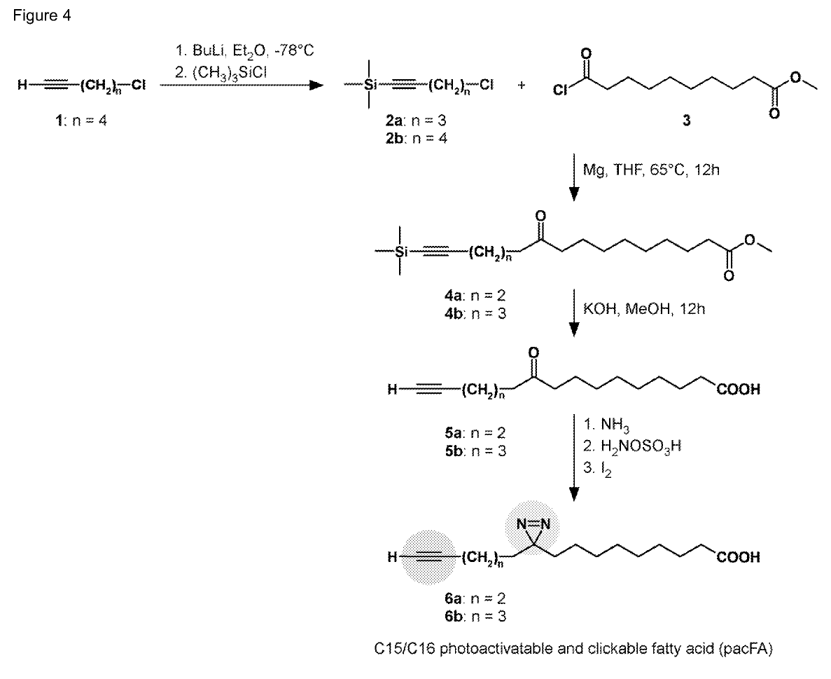

As an example of the synthesis of lipid precursors, herein the synthesis of fatty acid derivatives is shown, featuring two functional groups: a photoactivatable diazirine ring as well as a terminal alkyne moiety (FIG. 4). The diazirine ring is the smallest existing photoactivatable group. The terminal alkyne serves as bio-orthogonal chemical reporter. The latter was defined by Prescher & Bertozzi as a “non-native, non-perturbing chemical handle that can be modified in biological samples through highly selective reactions.”^{[2]} In chemical biology, the latter is used as a tool for tagging and visualizing biomolecules. Here, the terminal alkyne can be derivatized employing a chemistry that is known as click chemistry.

### Example 2

### Click Chemistry

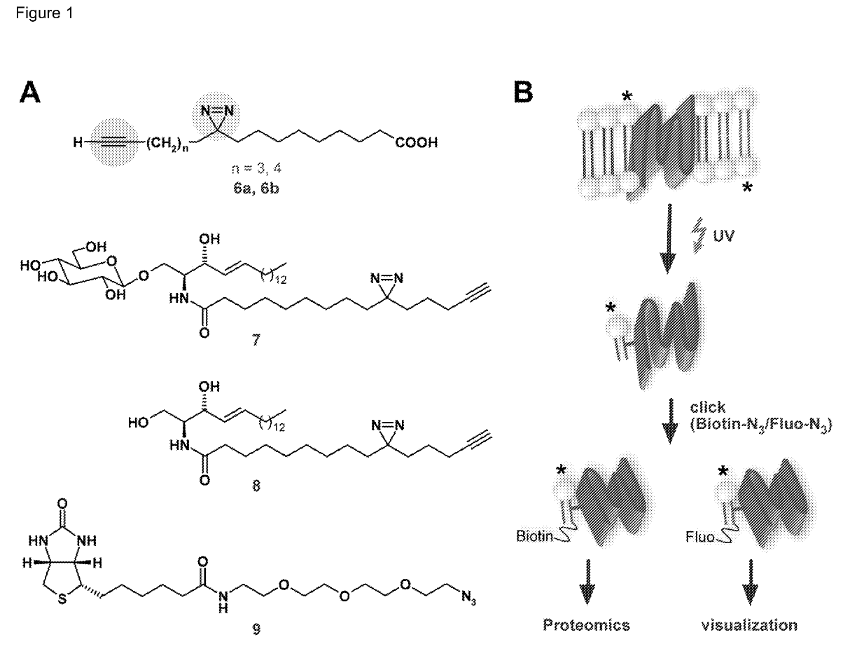

An example of click chemistry is the copper(I)-catalyzed azide-alkyne cyclo-addition leading to 1,2,3-triazoles—a reaction that is referred to as the “cream of the crop” of click reactions.^{[3]} The 15 C atom long photoactivatable and clickable fatty acid (C15pacFA) was obtained by a short three-step synthesis, which may be easily established in standard biochemical laboratories (see FIG. 4). The synthesis allows varying the chain length and positioning of the photoactivatable group. The fatty acids serve as a building block for the bio- as well as for the chemical synthesis of photoactivatable and clickable (pac) lipids and, therefore, provide fast access to a great number of bifunctionalized lipids. In addition to safety issues, the advantage towards radioactive photoactivatable lipid precursors is given by a rapid and highly sensitive readout. Even more important, cross-linked products can be purified after their respective derivatization. The approach is illustrated in FIG. 1 and can be dissected into two steps: (i) snap-shots of protein-lipid interactions are taken by means of photo-affinity labeling. (ii) Cross-linked products are labeled with a reporter molecule, e.g., alexa488-azide or biotin-azide, using click chemistry.

### Example 3

### Labeling of BSA

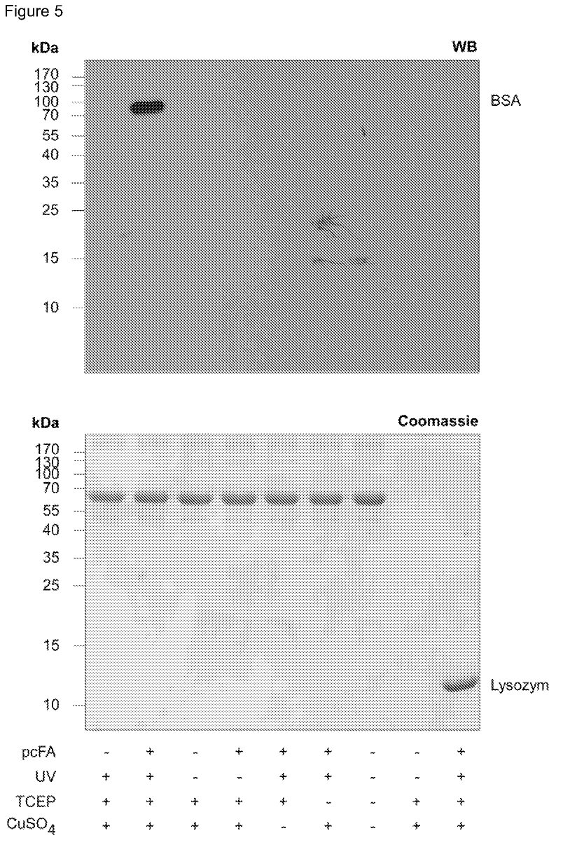

Serum albumin binds and transports fatty acids. As a proof-of-concept that C15pacFA can be used to tag proteins with an affinity for fatty acids, defatted bovine serum albumin (BSA) was incubated with C15pacFA and subsequently UV-irradiated. Thereafter, the mixture was subjected to a click reaction, applying biotin-azide (see FIG. 5). To demonstrate that photo-affinity labeling requires a specific interaction of the protein with the fatty acid, lysozyme, a protein known not to interact with lipids, was subjected to the same conditions. While BSA could be labeled after incubation with C15pacFA, UV-irradiation and subsequent click reaction with biotin-azide, no labeling of lysozyme was observed. CuSO₄ serves as a source for Cu(I) that is required for the catalytic cycle of the [3+2] cyclo-addition and was formed in situ by the addition of the reducing agent TCEP. Tagging of cross-linked BSA required the presence of the reducing agent TCEP. Therefore, we conclude that labeling of BSA depended on the click reaction. Further, we show that the presence of cross-linked products was a prerequisite for further derivatization by means of click chemistry. Significant labeling of cross-linked products was obtained after 1 hour at room temperature.

### Example 4

### Biocompatibility of pacFAs

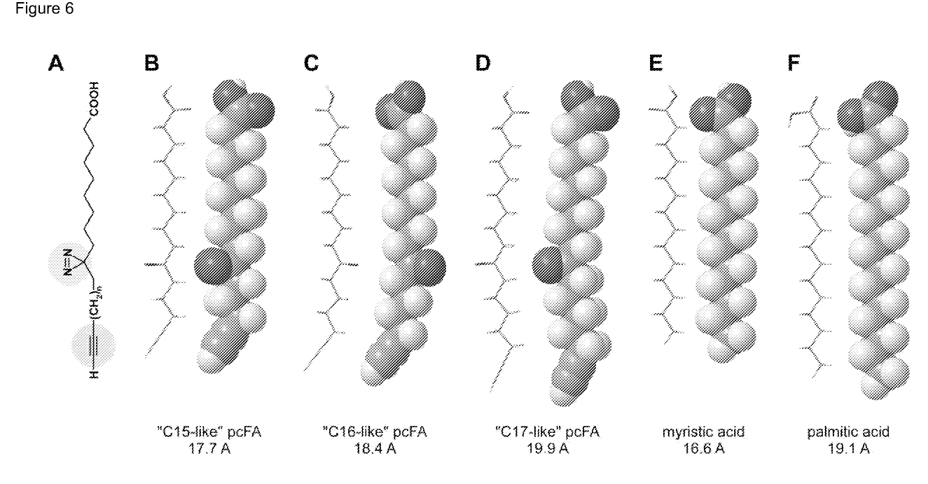

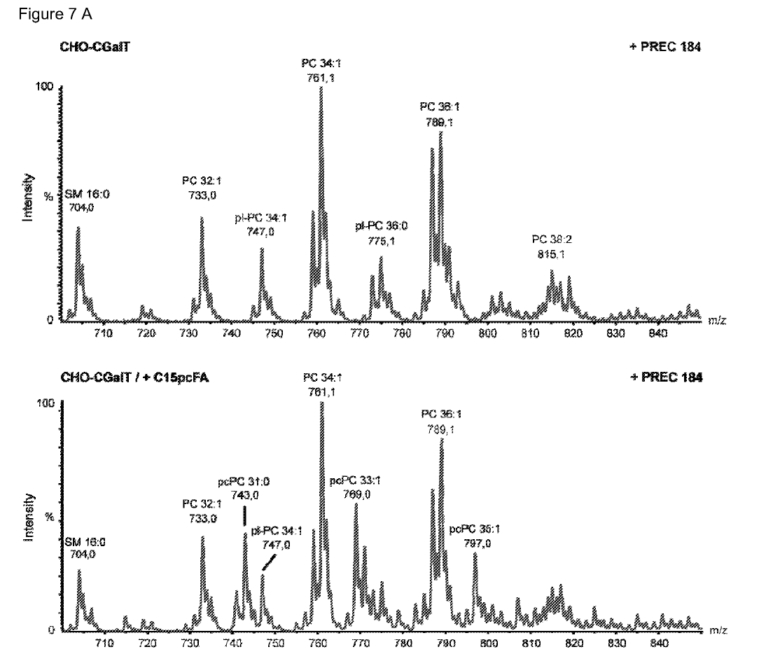

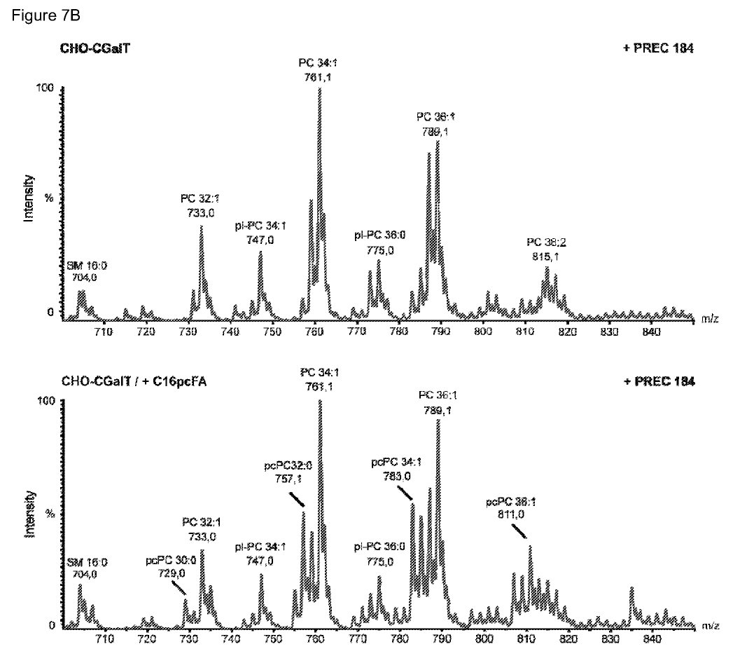

Given the small size of fatty acids, additional functional groups might have an impact on their physical properties. Space filling models are provided in FIG. 6. Due to the rigidity of the triple bond pacFAs are slightly kinked in the end and their overall length is shorter compared with their corresponding natural counterparts. In order to demonstrate the biocompatibility of C15/C16pacFA, lipid extracts of cells labeled with pacFAs were prepared and analyzed using tandem electrospray mass spectrometry (ESI-MS/MS) (see FIGS. 7A and 7B). Phosphatidylcholine (PC) is the major lipid class in eukaryotic cells. To analyze choline phosphate containing species, a precursor ion scanning procedure was employed selecting for fragments with a mass of 184 Da. Labeling of CHO-CGalT cells with C15pacFA led to additional peaks corresponding to pacPC species demonstrating that pacFAs enter the enzymatic machinery required for the biosynthesis of PC. UV irradiation of the lipid extracts gave rise to peaks with a mass 28 Da lower than the corresponding pacPC species due to the release of nitrogen followed by either an α-hydrogen migration or an intramolecular crosslink of the carbene (data not shown).^{[4]} No significant incorporation of C15pacFA into sphingomyelin (SM) was observed. SM16:0 is the major SM species in CHO cells. Its biosynthesis requires two palmitoyl-CoA: (i) as a precursor of the sphingoid backbone and (ii) for its N-acylation. The length of the N-acylated fatty acid is strictly controlled since there is no SM14:0 and only a minor amount of SM18:0 in CHO cells.^{[5]} Therefore, we assumed that the chain length of C15pacFA might interfere with its incorporation into SM. However, no incorporation of C16pacFA into SM was observed either showing that neither fatty acid is a substrate for SM biosynthesis. Similar results were obtained using mouse fibroblasts or Hela cells (data not shown). No morphological differences were observed after labeling with C15/C16pacFA. Intensities of the peaks corresponding to pacPC might be due to remodeling and/or differences in the ionization of pacPC species.

### Example 5

### Incorporation of pacFAs In Vivo

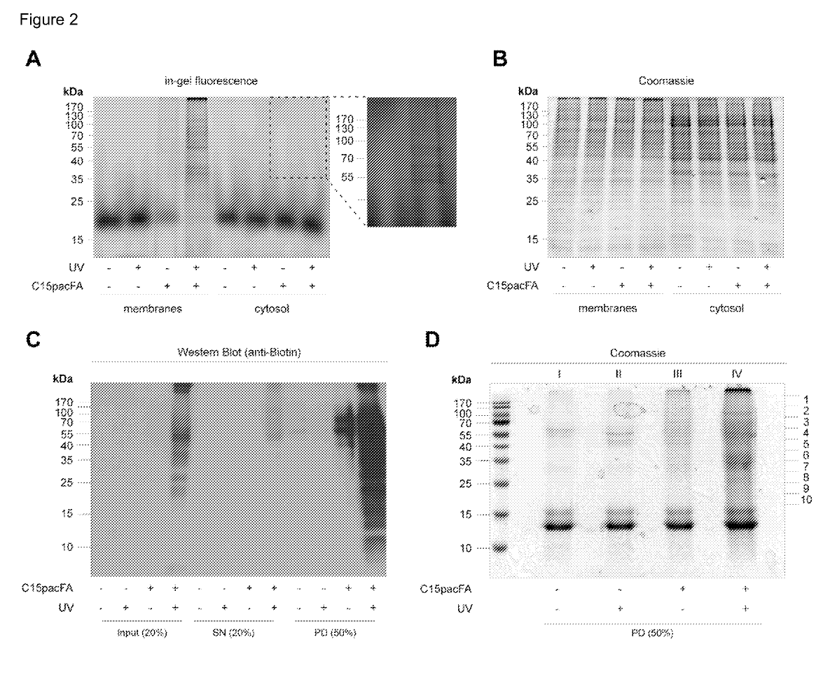

In order to see whether pacFAs can be used to investigate protein-lipid interactions in vivo, we administered C15pacFA to cells. After UV irradiation, cells were lysed and cytosol was separated from total membranes. Both membranes and cytosol were subjected to click reaction conditions and samples were analyzed by SDS-PAGE and in-gel fluorescence (FIG. 2). Notably, not only membrane proteins were labeled by means of photo-affinity labeling but also cytosolic proteins (see FIG. 2, Panel A). A specific set of proteins were labeled that were distinct from Coomassie-stained proteins (see FIG. 2, Panel B). Biotinylation of cross-linked products enabled their purification as shown in FIG. 2, Panel C). Purified proteins were separated on SDS-gels and subjected to mass spectrometry (FIG. 2, Panel D). 208 proteins were identified that were exclusively detected upon labeling cells with C15pacFA (FIG. 2, Panel D, lane III). A complete list of these proteins is given in Table 1. They most likely represent lipid-modified proteins. A Swiss-Prot database search revealed 11% of these proteins to have known lipid modifications like palmitoylation and myristoylation. Recently, Martin et al. made use of 17-octadecynoic acid to screen for palmitoylated proteins in Jurkat-T cells.^{[6]} 21% of the lipid-modified proteins identified here matched the palmitoylated proteins identified by Martin et al. Lipid-modified proteins could be assigned to multiple cellular organelles (Table 6). Proteins that were exclusively detected upon labeling with C15pacFA and subsequent cross-linking (lane IV) are given in Table 2. These proteins were labeled due to an interaction with a respective pac-lipid (FIG. 2, Panel C, lane 4). The proteins are constituents of various organelles as illustrated in Table 6.

A high number of these proteins are associated with transport and lipid metabolism. 14% of those proteins have been described as cytosolic proteins. The fact that the photoactivatable group is embedded within the hydrophobic core of the bilayer, cytosolic or peripheral membrane proteins can only be labeled if the protein dips into or extracts a photoactivatable and clickable lipid from the bilayer. This subset of proteins with a cytoplasmic localization is given in Table 3. Some of these proteins revealed (potential) transmembrane spans or lipid modifications like prenylation that allow interactions with lipids without their extraction. However, some proteins do not have potential transmembrane spans or post-translational lipid modifications suggesting that these proteins dip into cellular membranes and/or extract a lipid from them. One of the identified proteins is the well characterized phosphatidylinositol transfer protein β (PITPβ).

### Example 6

### Labeling of Cer and GlcCer Handling Proteins

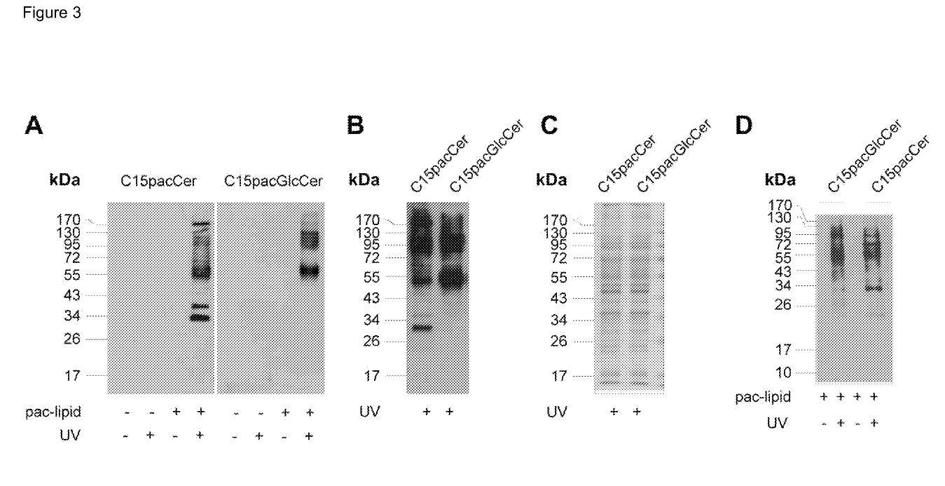

The sphingolipids sphingosine, sphingosine-1-phosphate, ceramide and glucosylceramide are bioactive lipids that contribute to a multitude of key cellular and pathological processes, including apoptosis, growth control, inflammatory processes and drug resistance in cancer. Ceramide and its interacting proteins are clinically relevant as they are recognized as determinants that improve sensitivity of cancer cells to radiation and chemotherapeutics. We identified ceramide- (Cer) and glucosylceramide- (GlcCer) interactive proteins in an in vitro screen employing their bifunctional analogues C15pacGlcCer (7) and C15pacCer (8) that were obtained by N-acylation of sphingosine and glucosylsphingosine with C15pacFA (FIG. 3, for structures of 7 and 8 see FIG. 1, Panel A). Liposomes containing either of the pac-lipids served as donor membranes for cytosolic proteins. Cytosol was isolated from the mouse melanoma glycosphingolipid-deficient GM95 cell line.^{[7]} Upon photo-affinity labeling cross-linked products were biotinylated and purified using NeutrAvidin beads. The mass spectrometric analysis of two independent experiments revealed 67 high-confidence ceramide interacting proteins, including the ceramide transfer protein CERT or COL4A3BP.^{[10]} A list of identified proteins can be found in Table 4. Subcellular localization of the identified proteins can be found in Table 5.

### Example 7

### Materials and Methods

10-azi-stearic acid (10-ASA) was synthesized as previously described.^{[11]} 11-Azido-3,6,9-trioxaundecan-1-amine (Sigma, 17758), (+)-Biotin N-hydroxysuccinimide ester (Sigma, H1759), chlorotrimethylsilane (Fluka, 92360), 1-chloro-5-trimethylsilyl-4-pentyne (Aldrich, 595918), 6-chloro-1-hexyne (ACROS, 380810250), GelCode Blue Stain Reagent (Pierce, 24590); hydroxylamine-O-sulfonic acid (Sigma-Aldrich, 55495-25G), absolute methanol (MeOH) over molecular sieve (Sigma-Aldrich, 65542-250 ml), methyl 10-chloro-10-oxodecanoate (Sigma-Aldrich, 401730-5G), Monoclonal Mouse Anti-Biotin Horseradish Peroxidase Conjugate (Jackson, 200-032-211), NeutrAvidin™ Agarose Resin (Thermo Scientific), SUPERSIGNAL® West Pico Chemi luminescent Substrate (Thermo Scientific, 34080), TBTA (tris(benzyltriazolylmethyl)amine, TBTA, Sigma, 678937), TCEP (Tris(2-carboxyethyl)phosphine hydrochloride, Sigma, 646547).

Chromatography was carried out using Merck silica gel 60. Thin layer chromatography (TLC) was performed using TLC Silica gel 60 F254 (MERCK, 1.05715.0001). Spots were detected using iodine or 20% sulfuric acid (v/v) and subsequent heating to 120° C. In addition to iodine, orcinol staining was applied for TLC analysis of C15pacGlcCer (0.3 g orcinol in 100 ml 2 M H₂SO₄, incubation at 130° C. for 5-10 minutes). All reagents were used as received.

Products were characterized by NMR (¹H, ¹³C) and high resolution mass spectrometry. NMR spectroscopic measurements were conducted on a Varian Oxford AS400 spectrometer at 25° C. and chemical shifts are given in ppm referenced to the residual solvent peak.

3×SDS-PAGE sample buffer (3×SB). 3×SB was prepared according to “Lab FAQs—Find a Quick Solution” by Roche, 3^{rd} Edition. Briefly, for 100 ml 15 ml 1.5 M Tris (pH 6.8), 9 ml 20% SDS, 45 ml glycerol and 22.5 ml β-mercaptoethanol and 2.7 mg bromphenol blue were mixed and the volume was adjusted to 100 ml with H₂O. Mixture was aliquoted and stored at −20° C.

CHCl₃/MeOH precipitation of proteins. To 200 μl sample 480 μl MeOH and 160 μl CHCl₃ were added. After vortexing 640 μl H₂O were added and the sample was spun 14,000 rpm, 5 minutes, 4° C. The upper layer was removed, 300 μl MeOH added and precipitated protein was collected by centrifugation (14,000 rpm, 30 minutes, 4° C.). The pellet was immediately resuspended in PBS containing 1% SDS.

Western Blot analysis of biotinylated proteins. The PVDF membrane was blocked with 3% milk in PBS for 2 hours. The membrane was shortly washed with PBS followed by incubation with monoclonal mouse anti-biotin coupled to horse radish peroxidase (1:1000 in PBS) for 1 hour. The membrane was washed three times for 10 minutes with PBS. The SUPERSIGNAL® West Pico Chemiluminescent Substrate system (Thermo Scientific) was used to detect biotinylated proteins.

Labeling of cytosol with C15pacCer/GlcCer. Cytosol from GM95 cells was prepared from ten 15-cm-dishes in a total volume of 4 ml lysis buffer as described above (protein concentration: ˜1 mg/ml). 1 ml of a liposome stock solution in PBS (egg-PC:pac-lipid 95:5 mol %) was prepared (total lipid concentration 10 mM). Briefly, lipids were mixed, dried under vacuum while rotating. PBS was added, followed by vigorous vortexing and subsequent sonification at 4° C. (microtip, 20 times for 1 second, output level 6). To 400 μl of cytosol 400 μl of the liposome solution were added (corresponding to 0.2 μmol of pac-lipid). The mixture was incubated for 30 minutes at room temperature while rotating followed by UV-irradiation. To remove lipids, samples were precipitated by CHCl₃/MeOH precipitation. The resulting pellet was immediately resuspended in 400 μl PBS containing 1% SDS. For complete solubilization samples were incubated for 10 minutes at 70° C. and subsequently subjected to click reactions as described. For analysis, 15 μl were subjected to SDS-PAGE and Western blotting. To enrich for biotinylated proteins, 300 μl of the click reactions were subjected to CHCl₃/MeOH precipitation followed by solubilization in 200 μl PBS/1% SDS. Samples were incubated at 70° C. for 10 minutes followed by sonification in a water bath for 16 minutes. CHCl₃/MeOH precipitation and solubilization were repeated once. 800 μl of PBS were added to achieve a final SDS concentration of 0.2%. Aggregates were removed by centrifugation (14,000 rpm, 1 minute, room temperature). 800 μl of the supernatant (input) were added to 50 μl settled, in PBS equilibrated, NeutrAvidin beads followed by incubation for 1 hour at room temperature while rotating. Beads were washed three times for 10 minutes with 1 ml of PBS/0.1% SDS). Biotinylated proteins were recovered from beads by addition of 30 μl 3×SB and incubation for 5 minutes at 95° C. (pull down). 200 μl of input as well as the supernatant after binding to NeutrAvidin beads (SN) were subjected to CHCl₃/MeOH precipitation. Protein pellets were solubilized in 40 μA 3×SB. 50% of input, 50% of SN and 25% of the pull down were applied to SDS-PAGE. 50% of the enriched proteins were applied on a second SDS-PAGE. Each lane was dissected into ten pieces and analyzed my mass spectrometry.

### Example 8

### Chemical Synthesis

Bold numbers refer to the compounds as illustrated in FIG. 4.

Synthesis of 1-Chloro-6-trimethylsilyl-5-hexyne (2b). 1-Chloro-6-trimethylsilyl-5-hexyne was synthesized as previously described.^{[12]} Briefly, 0.1 mol (12.12 ml) 6-Chloro-1-hexyne (1) was dissolved in 100 ml of dry ether, cooled to −78° C. and n-butyllithium (62.5 ml, 1.6 M) was added within 30 minutes. The reaction mixture was stirred at −78° C. for 2 hours. Chlorotrimethylsilane (16.5 ml, 0.13 mol) was added dropwise and the reaction mixture was brought to room temperature. After stirring overnight the precipitated salt was filtered and the solvent was removed by distillation. Continued distillation yielded 10.62 g (56.26 mmol, 56%) of 1-chloro-5-(trimethylsilyl)-5-hexyne (2b). ¹H NMR (CDCl₃) δ=3.55 (t, 2H, CH₂), 2.25 (t, 2H, CH₂), 1.89-1.86 (m, 2H, CH₂), 1.72-1.64 (m, 2H, CH₂), 0.14 (s, 9H, (CH₃)₃Si). ¹³C NMR (CDCl₃) δ=106.7, 85.4, 44.7, 31.7, 25.9, 19.3, 0.3 ppm.

Synthesis of 10-oxo-15-trimethylsilanyl-pentadec-14-yonic acid methyl ester (4a). The magnesium derivative of 2a was synthesized as previously described.^{[13]} The reaction was performed under nitrogen atmosphere. Briefly, 621.4 mg (25.56 mmol) magnesium turnings were covered with 10 ml dried THF. The mixture was heated to 50° C. Eight drops of dried and distilled 1,2-dibromoethane were added in order to activate the magnesium. 0.37 ml (0.82 g, 4.34 mmol) of 1,2-dibromethane were added to a solution of 3.92 ml (3.83 g, 21.30 mmol) of 1-chloro-5-trimethylsilyl-4-pentyne (2a) in 10 ml THF. The mixture was added dropwise within 1.5 hours to the magnesium turnings while stirring at 50° C. After addition, the reaction was stirred at 50° C. overnight to yield a transparent Grignard solution. 4a was synthesized as follows: The Grignard solution was added within 10 minutes to a solution of 5 g (21.30 mmol) 3 in 40 ml THF at 4° C. The mixture was stirred overnight at room temperature and then added to 200 g ice. The pH was adjusted to pH 1 by adding 1 N HCl. The organic phase was separated and the water phase was extracted four times with 100 ml diethyl ether. The combined organic phases were dried with Na₂SO₄ and purified by silica chromatography using hexane/diethyl ether 9:1 to yield 4a (5.4 g, 15.97 mmol, 75%). ¹H NMR (400 MHz, CDCl₃) δ=3.65 (s, 3H, CH₃O), 2.52 (t, J=7.2, 2H), 2.39 (t, J=7.2, 2H), 2.28 (t, J=7.6, 2H), 2.24 (t, J=6.8, 2H), 1.76 (quintet, J=6.8, 2H), 1.62-1.53 (m, 4H, CH₂), 1.28 (s, 8H, CH₂), 0.13 (s, 9H); ¹³C NMR (100 MHz, CDCl₃) δ=210.8, 174.4, 106.6, 85.5, 51.6, 43.1, 41.2, 34.2, 29.4, 29.3, 29.2, 25.1, 25.0, 24.0, 22.6, 19.3, 0.3 ppm. HRMS (m/z): [M+H]+calculated for C₁₉H₃₅O₃Si, 339.2355; found: 339.2352.

Synthesis of 10-oxo-16-trimethylsilanyl-hexadec-15-yonic acid methyl ester (4b). Synthesis was performed as described above. Purification by silica chromatography using hexane/ethylacetate 95:5 yielded 3.52 g, (9.98 mmol, 47%) of a transparent oil that crystallized at lower temperatures. NMR (400 MHz, CDCl₃): δ 3.63 (s, 3H), 2.34 (t, J=7.6 Hz, 2H), 2.32-2.18 (m, 6H), 1.70-1.48 (m, 8H), 1.27 (s, 8H), 0.12 (s, 9H); ¹³C NMR (100 MHz, CDCl₃): δ 212.2, 174.4, 157.2, 138.9, 51.6, 43.9, 34.3, 33.4, 33.2, 29.5, 29.5, 29.3, 29.3, 26.5, 26.1, 25.1, 23.7, −0.2; HRMS (m/z): [M+H]+calculated for C₂₀H₃₆O₃Si, 353.2512; found, 353.2495.

Synthesis of 10-oxo-pentadec-14-ynoic acid (5a). 0.6 g (1.77 mmol) of 4a were dissolved in 50 ml MeOH. 3 g of KOH were added and the mixture was refluxed over night. The solvent was evaporated and the residue was dissolved in 50 ml H₂O. The pH was adjusted to pH 1 by adding concentrated HCl. The water phase was extracted four times with 50 ml diethyl ether. The organic phase was dried over Na₂SO₄. The solvent was evaporated and the residue was purified by silica chromatography using hexane/diethyl ether/acidic acid 5:5:0.01 yielding 300 mg (1.19 mmol) of 5a (67%). R_{f} (hexane/diethyl ether/acidic acid 5:5:0.1)=0.54. ¹H NMR (400 MHz, CDCl₃) δ=2.53 (t, J=7.2, 2H), 2.38 (t, J=5.2, 2H), 2.32 (t, J=7.6, 2H), 2.20 (td, J=7.7, 2.8, 2H), 1.94 (t, J=2.8, 1H), 1.76 (quintet, J=6.8, 2H), 1.65-1.48 (m, 4H), 1.35-1.20 (m, 8H); ¹³C (100 MHz, CDCl₃) δ=210.9, 180.0, 83.9, 69.2, 43.2, 41.3, 34.2, 29.4, 29.4, 29.3, 29.2, 24.8, 24.0, 22.5, 18.0 ppm; HRMS (m/z): [M+H]⁺ calculated for C₁₅H₂₅O₃, 253.1803; found, 253.1768.

Synthesis of 10-oxo-hexadec-15-ynoic acid (5b). Synthesis was performed as described above. The product was purified by silica chromatography using hexane/diethyl ether/acidic acid 5:5:0.1 yielding a white solid.

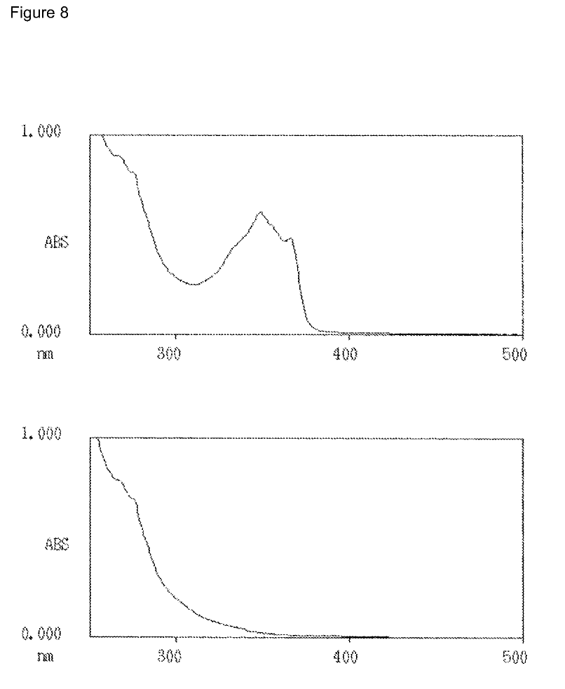

Synthesis of 9-(3-pent-4-ynyl-3-H-diazirin-3-yl)-nonanoic acid (15 CpacFA, 6a). The photoactivatable group was introduced as described.^{[11, 14, 15]} The reaction was performed under nitrogen atmosphere. 1 g (4 mmol) of keton 5a were dissolved in 100 ml dry MeOH and the mixture was cooled to 4° C. Ammonia gas was bubbled through the mixture till the solution was saturated (−3 h). A solution of 2.2 eq (8.8 mmol, 1 g) hydroxylamine-O-sulfonic acid in 10 ml dry MeOH was added within 5 minutes. Thereby, the mixture got slightly turbid. After 30 minutes stirring at 4° C. the mixture was stirred for 4 hours at room temperature. The solution was filtered to remove precipitated (NH₄)₂SO₄. 5 ml triethylamine were added and the solvent was evaporated. The residue was dissolved in 40 ml MeOH and 10 ml triethylamine. 1 g of iodine was dissolved in 10 ml MeOH and added dropwise until the yellow color persisted. The solvent was evaporated and the residue was dissolved in 100 ml ethylacetate. The mixture was extracted with 50 ml H₂O. The water phase was extracted three more times with 100 ml ethyl acetate. The combined organic phases were dried over Na₂SO₄ and purified by silica chromatography using hexane/ethylacetate/acidic acid 96:4:1 as solvent system. Note: (i) The approximate yield was photometrically determined. Fractions containing the product were identified by the characteristic absorption of the diazirine group at 349 and 367 nm. Wavelength scans of a solution of compound 6a in ethanol before (upper panel) and after UV-irradiation (lower panel) are provided in FIG. 8. Yield: 174 mg (658 μmol, 16%). Note: ˜37% of non-converted ketone were recovered. TLC (hexane:diethyl ether:acidic acid, 5:5:0.1 v/v): R_{f}=0.59; ¹H NMR (400 MHz, CDCl₃) δ=2.34 (t, J=7.6, 2H), 2.16 (td, J=7.2, 2.8, 2H), 1.94 (t, J=2.8, 1H), 1.62 (quintet, J=7.2, 2H), 1.50-1.46 (m, 2H), 1.38-1.18 (m, 12H), 1.11-1.03 (m, 2H); ¹³C NMR (100 MHz, CDCl₃): δ 180.6, 83.7, 69.1, 34.3, 33.0, 32.0, 29.3, 29.3, 29.3, 29.2, 28.6, 24.8, 24.0, 23.0, 18.2; HRMS (m/z): [M+Na]+calculated for C₁₅H₂₄N₂O₂, 287.1735; found, 287.1711.

Synthesis of 9-(3-Pent-4-ynyl-3-H-diazirin-3-yl)-nonanoic acid (C16pacFA, 6b). Synthesis was performed as described above. 690 mg of ketone (2.59 mmol) were subjected to the reaction and purified by silica chromatography using hexane/ethylacetate/acetic acid 92:8:1 as a solvent system. Yield: 250 mg (898 μmol, 34.67%). Note: 43.5% (300 mg) of non-converted ketone were recovered. ¹H NMR (CDCl₃) δ=2.33 (t, 2H), 2.14 (dt, 2H), 1.93 (t, 1H, CCH), 1.65-1.57 (m, 2H), 1.53-1.43 (m, 2H), 1.38-1.15 (m, 14H), 1.1-1.0 (m, 2H); HRMS (m/z): [M+Na]+calculated for C₁₆H₂₆N₂O₂, 301.1892; found, 301.1884.

Synthesis of photoactivatable and clickable glucosylceramide (C15pacGlcCer, 7). C15pacGlcCer (7) was synthesized according to Kishimoto et al.^{[16]} Briefly, 10.9 μmol (5 mg) 1-β-D-glucosylsphingosine (GlcSph), 10.9 μmol C15pacFA (2.9 mg), 21.8 mmol triphenylphosphine, and 21.8 μmol 2,2-dithiodipyridine were dissolved in 250 μl DMF. The mixture was shaken vigorously in the dark at room temperature overnight. 500 μl water were added and the mixture was freeze dried. The residue was dissolved in CHCl₃/MeOH 2:1 and purified by preparative thin layer chromatography using Et₂O/hexane/AcOH, 80:30:5 (v/v). The origin was scraped and extracted with CHCl₃/MeOH 2:1 (v/v). The extract was purified by an additional preparative thin layer chromatography using CHCl₃/acetone/MeOH/AcOH/H₂O 50:20:10:10:5 yielding 4.7 mg of C15pacGlcCer (6.64 μmol, 61%). TLC (CHCl₃/acetone/MeOH/AcOH/H₂O, 50:20:10:10:5 v/v): R_{f} (C15pacGlcCer)=0.73, R_{f} (GlcSph)=0.16, R_{f} (C15pacFA)=0.96; HRMS (m/z): [M+Na]+calculated for C₃₉H₆₉N₃O₃, 730.4982; found, 730.4982; λₘₐₓ 349.5 nm.

Synthesis of photoactivatable and clickable ceramide (C15pacCer, 8). C15pacCer was synthesized analogous to C15pacGlcCer starting from 8.2 mg (27.3 mmol) sphingosine yielding 8.6 mg C15pacCer (15.8 mmol, 58%). C15pacCer was purified by preparative thin layer chromatography using Et₂O/hexane/AcOH 80:30:5 as solvent system. Eight bands were scraped and extracted with CHCl₃/MeOH 2:1. The extract that co-migrated with natural ceramide revealed the characteristic absorbance of the diazirine ring. TLC (Et₂O/hexane/AcOH, 80:30:5 v/v): R_{f}=0.66; HRMS (m/z): [M+Na]+calculated for C₃₃H₅₉N₃O₃, 568.4454; found, 568.4464; λₘₐₓ 349.5 and 366.5.

Synthesis of biotin-azides (9). The reaction was performed under nitrogen atmosphere. 23.5 mg (68.86 mmol) (+)-biotin N-hydroxysuccinimide ester and 37.5 triethylamine were dissolved in 3 ml dry MeOH. After 30 minutes of stirring, 35 μl of 11-Azido-3,6,9-trioxaundecan-1-amine were added and the mixture was stirred for 48 hours at room temperature. The solvent was evaporated and the residue was purified two times by silica chromatography. First, acetone/hexane 4:1 was used as solvent system. In a second purification step CHCl₃/MeOH 65:10 was applied to obtain white crystals of biotin azide. ¹H NMR (CD₃OD) δ=4.51 (m, 1H, CH-1-Biotin), 4.32 (m, 1H, CH-4-Biotin), 3.70-3.63 (m, 8H, O(CH₂CH₂O)—PEG, 3.39 (m, 4H, CH₂NH and CH₂N₃—PEG), 3.20 (m, 1H, CH-3-Biotin), 2.93 (dd, 1H, J=4.8, 12.8 Hz, CH-2a-Biotin), 2.71 (m, 1H, CH-2b-Biotin), 2.23 (t, 1H, J=7.6 Hz, CH₂CO-Biotin), 1.76-1.43 (m, 6H, (CH₂)₃-Biotin).^{[17]} MALDI [M+H]⁺: m/z=445.5866 Da.

### Example 9

### Cell Biological Experiments

CHO-CGaIT cells have been described previously.^{[18]} Cells were grown in DMEM, stable glutamine, 4.5 g/liter glucose and 10% FCS at 37° C. with 5% CO₂. CHO-CGalT were grown in the presence of 10 μg/ml geneticin (G418). Labeling experiments were performed in DMEM supplemented with delipidized FCS (charcoal/dextran treated fetal calf serum from Thermo Scientific, 29202; HyClone, SH30068.02).

### Example 10

### Photo-Affinity Labeling

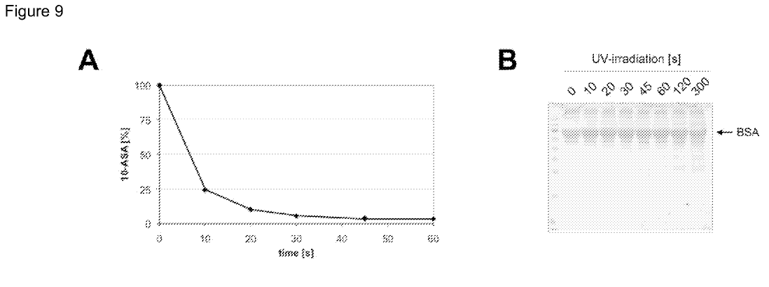

Samples were irradiated applying a 200 W high pressure mercury lamp (Oriel Photomax) equipped with a PYREX® glass filter to remove wavelengths below 350 nm. Samples were placed on ice at a distance from 35 cm from the light source. In order to optimize cross-linking conditions we made use of the characteristic absorption of the diazirine group (FIG. 8). An ethanolic solution of 10-azi stearic acid (10-ASA) was irradiated under the conditions described above and the loss of absorption at 349.5 nm was monitored (FIG. 9, Panel A). Thirty seconds of irradiation activated 95% of the photoactivatable group. Samples were UV irradiated for 1 minute. To exclude degradation of proteins under these conditions a 1 mg/ml solution of BSA was irradiated up to 5 minutes and analyzed by gel electrophoresis and subsequent Coomassie staining (FIG. 9, Panel B). While major degradation of protein was observed within seconds employing a glass filter to remove wavelengths below 200 nm (data not shown), no degradation of protein was observed upon 5 minutes of irradiation with the glass filter to remove wavelengths below 350 nm. We investigated whether terminal alkyne groups tolerate the UV irradiation conditions used in this study by irradiation of 3-phenyl-1-propyne for 5 minutes. ¹H-NMR analysis revealed the stability of alkynes towards UV irradiation under these conditions (data not shown).

### Example 11

### Click Reaction

The following stock solutions were prepared and stored at −20° C.: 25 mM TCEP/KOH, pH 7.5 in H₂O (reducing agent), 2.5 mM tris(benzyltriazolylmethyl)amine (TBTA) in DMSO (ligand; stabilizes Cu(I) towards disproportion and oxidation),^{[19]} 25 mM CuSO₄ in H₂O, 25 mM biotin azide in DMSO, 2 mM alexa-azide in DMSO. The click reaction was performed in PBS containing 1% SDS by the stepwise addition of (i) TCEP (freshly thawed; 1 mM), (ii) TBTA (0.1 mM), (iii) CuSO₄ (1 mM) and (iv) biotin azide (1 mM) or alexa azide (80 μM). Final concentrations are given in brackets. Samples were incubated for 1 hour at 37° C. and subsequently subjected to analysis or purification. For purification, a CHCl₃/MeOH precipitation of the proteins was used to remove free biotin-azide as well as biotinylated lipids (see below). For complete removal samples were subjected twice to CHCl₃/MeOH precipitation.

### Example 12

### Labeling of Delipidated BSA with C15pacFA

Solutions of delipidated BSA (1 mg/ml; approximately 15 nmol/ml) and lysozyme (1 mg/ml) were prepared. 90 nmol of C15pacFA were added to 1 ml of each solution in form of an ethanolic solution under vigorous stirring. Final EtOH concentration was below 0.5%. Sample was incubated for 1 hour at room temperature while stirring and subsequently subjected to UV irradiation as indicated (see FIG. 5). Samples were adjusted to final concentration of 1% SDS by addition of a 10% SDS stock solution. After 1 hour incubation at room temperature samples were subjected to click reactions as described above.

### Example 13

### Labeling of Cells with pacFA

Typically, 10 cm dishes of cells (approximately 5×10⁶ cells) were labeled overnight with 100 μM pacFA in medium supplemented with 10% delipidated fetal calf serum (1 μmol pacFA/10 ml medium). The fatty acid was added to the medium from an ethanolic stock solution (final ethanol concentration 0.2%). Cells were washed with 5 ml PBS, overlaid with 5 ml PBS followed by UV irradiation where indicated. PBS was removed and the cells were scraped in 1 ml PBS. Cells were collected (14,000 rpm, 5 minutes, 4° C.) and either subjected to lipid extraction followed by lipid analysis by means of ESI-MS/MS, or total membranes and cytosol were prepared as described below. Total membranes as well as cytosolic fractions were subjected to click reactions with alexa488-azide.

### Example 14

### Preparation of Membrane and Cytosolic Fractions

Cells of one 10 cm dish were resuspended in 500 μl lysis buffer (50 mM Tris, pH 6.8; 1 mM EDTA, 0.3 M sucrose; 1 mM PMSF; protease inhibitor cocktail). Cells were homogenized by passing them 20 times through a 26G1/2 needle using a 1 ml syringe. Nuclei were removed by centrifugation (600 g, 5 minutes, 4° C.). The supernatant was spun at 100,000 g for 60 minutes to collect membranes. Membranes derived were resuspended in 200 μl of PBS/1% SDS. Note: Samples can be stored at −20° C. For solubilization the sample was heated to 70° C. for 10 minutes followed by 16 minutes of sonification in a sonicating water bath. If necessary heating and sonification were repeated until the sample was solubilized. The sample was diluted with PBS/1% SDS to a final volume of 800 μl and then subjected to the click reaction.

### Example 15

### Purification of Cross-Linked Products

CHCl₃/MeOH precipitates of click reactions yielded a light blue pellet that was resuspended in 200 μl PBS/1% SDS. To achieve solubilization, samples were heated to 70° C. for 10 minutes followed by sonification in a water bath, if necessary followed by heating and sonification one more time. The sample was diluted 1:5 with PBS to achieve a final concentration of 0.2% SDS. Insoluble material was removed by centrifugation (3,000 g, 3 minutes, room temperature). The supernatant was isolated from a turquoise pellet to achieve a transparent solution. 200 μl were kept as input while 800 μl were subjected to purification with NeutrAvidin beads. 100 μl NeutrAvidin beads were equilibrated by three washes with 1 ml PBS each. Beads were collected by centrifugation (100 g, 1 minute, room temperature). The sample was added to NeutrAvidin beads and incubated at room temperature for 1 hour. Note: SDS precipitates at 4° C. Beads were washed three times with 1 ml of PBS and bound proteins were eluted using sample buffer (SB). For this, 20 μl of 3×SB (see below) were added and beads were incubated for 30 minutes at room temperature. Note: during incubation supernatants were precipitated for analysis using CHCl₃/MeOH precipitation. NeutrAvidin beads were boiled for 5 minutes at 95° C. and the supernatant was removed.

### Example 16

### Lipid Extraction and Mass Spectrometric Analysis of Lipid Extracts

Lipids were extracted according to Bligh and Dyer and analyzed as described previously (K. Retra, O. B. Bleijerveld, R. A. van Gestel, A. G. Tielens, J. J. van Hellemond, J. F. Brouwers, Rapid Commun. Mass Spectrom. 2008, 22, 1853.^{[20]}

### Example 17

### LC-MS/MS

Gel lanes containing pulled down proteins were cut into 10 bands and proteins were reduced with 1,4-dithiothreitol (6.5 mM) and alkylated with iodoacetamide reagent (54 mM). After thorough washing, the pieces were rehydrated in trypsin solution (10 ng/μl) on ice. After addition of 30 μl of NH₄HCO₃ (50 mM, pH 8.5), samples were digested for 16 hours at 37° C. The supernatant of the digest was collected and the gel pieces were washed for 15 minutes in 5% formic acid at room temperature, after which the supernatant was combined with the earlier fraction and stored at −20° C. All LC-MS/MS analyses were performed on an LTQ-Orbitrap XL mass spectrometer (Thermo, San Jose, Calif.) connected to an Agilent 1200 series nano LC system. Peptides were separated on C18 with a multi-step gradient of 0.6% acetic acid (buffer A) and 0.6% acetic acid/80% acetonitrile (ACN) (buffer B). The mass spectrometer was operated in the data-dependent mode to automatically switch between MS and MS/MS. Raw MS data were converted to peak lists using DTASuperCharge. The spectra were searched with Mascot against all rodent proteins in the Swissprot (v56.2) database with a precursor mass tolerance of 50 ppm and a product mass tolerance of 0.6 Da with trypsin as an enzyme, allowing two miscleavages. Peptide identifications were accepted with a Mascot score greater than 30 and a p value smaller than 0.05, and proteins were identified with at least two unique peptides. Semi-quantitative analysis was done by spectral counting.

### Example 18

### Cell Imaging of Bifunctional Lipids

Microscopy of lipids requires a fluorescent tag on the lipid. In contrast to polyene lipids, most fluorescent tags interfere with the properties of thus labeled lipids.^{[24]} Recently Neef et al. demonstrated that terminal alkyne containing lipids can be used for their visualization upon derivatization by means of click chemistry.^{[25]} Lipids are highly dynamic and their chemical fixation remained difficult. Here, we demonstrate that pac-lipids can be used to fix lipids in the nano-second range followed by their visualization using click chemistry.^{[26]} To this end, C15pacFA was administered to cells. Lipids were fixed in vivo by UV-irradiation followed by the fixation of cells with methanol. In order to remove non-protein-crosslinked lipids, cells were extracted according to Bligh and Dyer.^{[27]} Samples were subjected to click reactions with alexa-azide and lipids were visualized by fluorescent microscopy. Fluorescent structures were only visible in the UV irradiated samples treated with C15pacFA.

### Example 19

### Fluorescence Labeling of Lipids

HeLa cells grown on cover slips in a 24-well plate were labeled for 1 hour in 0.5 ml DMEM 4.5 g/liter glucose supplemented with 10% delipidated FCS and 100 μM C15pacFA. The cells were washed with 0.5 mL PBS, overlaid with 0.5 mL PBS and UV irradiated on ice for 2 minutes. After UV irradiation the cells were fixed with 0.5 mL of −20° C. cold MeOH for 10 minutes. After fixation cells were subjected to one, two or three rounds of lipid extraction according to Bligh and Dyer using 0.5 ml of CHCl₃/MeOH/AcOH 10:55:0.75 v/v for each extraction (for each extraction coverslips were incubated for 1 minute at room temperature). The coverslips were washed with 0.5 mL PBS and then subjected to click reactions. To this end, each coverslip was covered with 48 μL of a freshly prepared solution of 0.86 mM TCEP, 86 μM TBTA, 0.86 mM CuSO4 and 18 μM alexa-488 azide in PBS. Samples were incubated for 1 hour at room temperature. Coverslips were washed three times with 0.5 mL PBS, two times with water and then were mounted using 5 μL of Vectashield (Vector Laboratories, H-1000).

### REFERENCES

[1] P. Haberkant, G. van Meer, Biol. Chem. 2009.
[2] J. A. Prescher, C. R. Bertozzi, Nat. Chem. Biol. 2005, 1, 13.
[3] H. C. Kolb, M. G. Finn, K. B. Sharpless, Angew Chem. Int. Ed. Engl. 2001, 40, 2004.
[4] D. A. Modarelli, S. Morgan, M. S. Platz, J. Am. Chem. Soc. 1992, 114, 7034.
[5] B. Brügger, R. Sandhoff, S. Wegehingel, K. Gorgas, J. Malsam, J. B. Helms, W. D. Lehmann, W. Nickel, F. T. Wieland, J. Cell Biol. 2000, 151, 507.
[6] B. R. Martin, B. F. Cravatt, Nat. Methods 2009, 6, 135.
[7] S. Ichikawa, N. Nakajo, H. Sakiyama, Y. Hirabayashi, Proc. Natl. Acad. Sci. U.S.A. 1994, 91, 2703.
[8] C. Swanton, M. Marani, O. Pardo, P. H. Warne, G. Kelly, E. Sahai, F. Elustondo, J. Chang, J. Temple, A. A. Ahmed, J. D. Brenton, J. Downward, B. Nicke, Cancer Cell 2007, 11, 498.
[9] Y. A. Hannun, L. M. Obeid, Nat. Rev. Mol. Cell Biol. 2008, 9, 139.
[10] K. Hanada, K. Kumagai, S. Yasuda, Y. Miura, M. Kawano, M. Fukasawa, M. Nishijima, Nature 2003, 426, 803.
[11] C. Thiele, M. J. Hannah, F. Fahrenholz, W. B. Huttner, Nat. Cell Biol. 2000, 2, 42.
[12] E. Koft, A. Smith, J. Org. Chem. 1984, 49, 832.
[13] J. Drouin, F. Leyendecker, J. M. Conia, Tetrahedron 1980, 36, 1203.
[14] R. F. Church, R. R. Maleike, M. J. Weiss, J. Med. Chem. 1972, 15, 514.
[15] P. Haberkant, O, Schmitt, F. X. Contreras, C. Thiele, K. Hanada, H. Sprong, C. Reinhard, F. T. Wieland, B. Brugger, J. Lipid. Res. 2008, 49, 251.
[16] Y. Kishimoto, Chem. Phys. Lipids 1975, 15, 33.
[17] X. L. Sun, C. L. Stabler, C. S. Cazalis, E. L. Chaikof, Bioconjug. Chem. 2006, 17, 52.
[18] P. van der Bijl, G. J. Strous, M. Lopes-Cardozo, J. Thomas-Oates, G. van Meer, Biochem. J. 1996, 317 (Pt 2), 589.
[19] T. R. Chan, R. Hilgraf, K. B. Sharpless, V. V. Fokin, Org. Lett. 2004, 6, 2853.
[20] E. G. Bligh, W. J. Dyer, Can. J. Biochem. Physiol. 1959, 37, 911.
[21] R. F. Church, R. R. Maleike, M. J. Weiss, J. Med. Chem. 1972, 15, 514.
[22] P. Haberkant, O, Schmitt, F. X. Contreras, C. Thiele, K. Hanada, H. Sprong, C. Reinhard, F. T. Wieland, B. Bragger, J. Lipid Res. 2008, 49, 251.
[23] R. Church, M. Weiss, J. Org. Chem. 1970, 35, 2465
[24] L. Kuerschner, C. S. Ejsing, K. Ekroos, A. Shevchenko, K. I. Anderson, C. Thiele, Nat. Methods 2005, 2, 39.
[25] A. B. Neef, C. Schultz, Angew Chem. Int. Ed. Engl. 2009, 48, 1498.
[26] F. Ford, T. Yuzawa, M. Platz, S. Matzinger, M. Fülscher, J. Am. Chem. Soc. 1998, 120, 4430.
[27] E. G. Bligh, W. J. Dyer, Can. J. Biochem. Physiol. 1959, 37, 911.

TABLE 1
Spectral count data of high-confidence proteins measured by tandem LC-MS from C15pacFA-labeled CHO-cells.
Roman numbers refer to the lane of the Coomassie stained gel in FIG. 2, Panel D.

| Nr | Protein | AccNr | kDa | I | II | III | IV |
|---|---|---|---|---|---|---|---|
| 39 | Epoxide hydrolase 1 OS = Rattus norvegicus GN = Ephx1 PE = 1 SV = 1 | HYEP_RAT | 53 | 0 | 0 | 3 | 41 |
| 42 | Prostaglandin F2 receptor negative regulator OS = Rattus norvegicus GN = Ptgfrn PE = 1 SV = 1 | FPRP_RAT | 99 | 0 | 0 | 29 | 16 |
| 53 | Cytochrome P450 51A1 OS = Rattus norvegicus GN = Cyp51a1 PE = 2 SV = 1 | CP51A_RAT | 57 | 0 | 0 | 1 | 45 |
| 57 | Mitochondrial carrier homolog 2 OS = Mus musculus GN = Mtch2 PE = 1 SV = 1 | MTCH2_MOUSE | 33 | 0 | 0 | 1 | 42 |
| 58 | Neural cell adhesion molecule 1 OS = Mus musculus GN = Ncam1 PE = 1 SV = 3 | NCAM1_MOUSE | 119 | 0 | 0 | 34 | 10 |
| 61 | Hexokinase-1 OS = Rattus norvegicus GN = Hk1 PE = 1 SV = 4 | HXK1_RAT | 102 | 0 | 0 | 4 | 43 |
| 72 | Peroxisomal multifunctional enzyme type 2 OS = Rattus norvegicus GN = Hsd17b4 PE = 1 SV = 3 | DHB4_RAT | 79 | 0 | 0 | 30 | 4 |
| 76 | 3-ketoacyl-CoA thiolase A, peroxisomal OS = Rattus norvegicus GN = Acaa1a PE = 2 SV = 2 | THIKA_RAT (+1) | 44 | 0 | 0 | 25 | 7 |
| 78 | Alkyldihydroxyacetonephosphate synthase, peroxisomal OS = Rattus norvegicus GN = Agps PE = 2 SV = 1 | ADAS_RAT | 72 | 0 | 0 | 27 | 14 |
| 92 | Non-specific lipid-transfer protein OS = Mus musculus GN = Scp2 PE = 1 SV = 3 | NLTP_MOUSE | 59 | 0 | 0 | 23 | 5 |
| 102 | Mitochondrial import inner membrane translocase subunit Tim23 OS = Mus musculus GN = Timm23 PE = 2 SV = 1 | TIM23_MOUSE | 22 | 0 | 0 | 3 | 25 |
| 109 | Signal recognition particle receptor subunit beta OS = Rattus norvegicus GN = Srprb PE = 2 SV = 1 | SRPRB_RAT | 30 | 0 | 0 | 5 | 20 |
| 110 | ATP synthase subunit b, mitochondrial OS = Mus musculus GN = Atp5f1 PE = 1 SV = 1 | AT5F1_MOUSE | 29 | 0 | 0 | 3 | 18 |
| 111 | Peroxisomal acyl-coenzyme A oxidase 1 OS = Mus musculus GN = Acox1 PE = 1 SV = 4 | ACOX1_MOUSE | 75 | 0 | 0 | 19 | 8 |
| 112 | Leucine-rich repeat-containing protein 59 OS = Mus musculus GN = Lrrc59 PE = 2 SV = 1 | LRC59_MOUSE (+1) | 35 | 0 | 0 | 2 | 24 |
| 113 | Synaptosomal-associated protein 23 OS = Mus musculus GN = Snap23 PE = 1 SV = 1 | SNP23_MOUSE (+1) | 23 | 0 | 0 | 15 | 8 |
| 116 | Caveolin-1 OS = Rattus norvegicus GN = Cav1 PE = 1 SV = 3 | CAV1_RAT | 21 | 0 | 0 | 5 | 18 |
| 117 | Dephospho-CoA kinase domain-containing protein OS = Mus musculus GN = Dcakd PE = 2 SV = 1 | DCAKD_MOUSE | 26 | 0 | 0 | 2 | 19 |
| 120 | Eukaryotic initiation factor 4A-1 OS = Mus musculus GN = Eif4a1 PE = 2 SV = 1 | IF4A1_MOUSE | 46 | 0 | 0 | 20 | 2 |
| 121 | Bone marrow stromal antigen 2 OS = Cricetulus griseus GN = Bst2 PE = 2 SV = 1 | BST2_CRIGR | 23 | 0 | 0 | 18 | 10 |
| 123 | Calcium-binding mitochondrial carrier protein Aralar2 OS = Mus musculus GN = Slc25a13 PE = 1 SV = 1 | CMC2_MOUSE | 74 | 0 | 0 | 5 | 19 |
| 128 | NADPH--cytochrome P450 reductase OS = Mus musculus GN = Por PE = 1 SV = 2 | NCPR_MOUSE | 77 | 0 | 0 | 2 | 20 |
| 135 | Signal peptidase complex catalytic subunit SEC11A OS = Mus musculus GN = Sec11a PE = 2 SV = 1 | SC11A_MOUSE | 21 | 0 | 0 | 1 | 17 |
| 139 | Extended-synaptotagmin-1 OS = Mus musculus GN = Fam62a PE = 2 SV = 2 | ESYT1_MOUSE | 122 | 0 | 0 | 5 | 18 |
| 140 | Cytochrome b5 type B OS = Mus musculus GN = Cyb5b PE = 1 SV = 1 | CYB5B_MOUSE | 16 | 0 | 0 | 9 | 17 |
| 141 | Polypyrimidine tract-binding protein 1 OS = Rattus norvegicus GN = Ptbp1 PE = 1 SV = 1 | PTBP1_RAT | 59 | 0 | 0 | 12 | 3 |
| 145 | Dynamin-like 120 protein, mitochondrial OS = Mus musculus GN = Opa1 PE = 1 SV = 1 | OPA1_MOUSE | 111 | 0 | 0 | 4 | 15 |
| 147 | Minor histocompatibility antigen H13 OS = Mus musculus GN = Hm13 PE = 1 SV = 1 | HM13_MOUSE | 42 | 0 | 0 | 1 | 20 |
| 148 | Medium-chain specific acyl-CoA dehydrogenase, mitochondrial OS = Mus musculus GN = Acadm PE = 1 SV = 1 | ACADM_MOUSE | 46 | 0 | 0 | 15 | 8 |
| 154 | Succinate dehydrogenase [ubiquinone] flavoprotein subunit, mitochondrial OS = Mus musculus GN = Sdha PE = 1 SV = 1″ | DHSA_MOUSE | 73 | 0 | 0 | 17 | 3 |
| 155 | Mitochondrial import receptor subunit TOM22 homolog OS = Mus musculus GN = Tomm22 PE = 2 SV = 3 | TOM22_MOUSE | 16 | 0 | 0 | 3 | 18 |
| 164 | Leucyl-cystinyl aminopeptidase OS = Mus musculus GN = Lnpep PE = 2 SV = 1 | LCAP_MOUSE | 117 | 0 | 0 | 15 | 3 |
| 165 | Erlin-1 OS = Mus musculus GN = Erlin1 PE = 2 SV = 1 | ERLN1_MOUSE | 39 | 0 | 0 | 1 | 20 |
| 173 | Guanine nucleotide-binding protein G(q) subunit alpha OS = Rattus norvegicus GN = Gnaq PE = 2 SV = 1 | GNAQ_RAT | 41 | 0 | 0 | 8 | 5 |
| 176 | Transmembrane emp24 domain-containing protein 1 OS = Mus musculus GN = Tmed1 PE = 2 SV = 1 | TMED1_MOUSE | 25 | 0 | 0 | 4 | 14 |
| 177 | Flotillin-1 OS = Mus musculus GN = Flot1 PE = 1 SV = 1 | FLOT1_MOUSE (+1) | 48 | 0 | 0 | 12 | 3 |
| 179 | Reticulon-3 OS = Rattus norvegicus GN = Rtn3 PE = 1 SV = 1 | RTN3_RAT | 102 | 0 | 0 | 1 | 6 |
| 184 | Ras-related protein Rab-18 OS = Mus musculus GN = Rab18 PE = 2 SV = 2 | RAB18_MOUSE (+1) | 23 | 0 | 0 | 9 | 13 |
| 185 | Transmembrane and coiled-coil domain-containing protein 1 OS = Mus musculus GN = Tmco1 PE = 2 SV = 1 | Endo | 21 | 0 | 0 | 3 | 14 |
| 197 | Protein FAM3C OS = Mus musculus GN = Fam3c PE = 1 SV = 1 | FAM3C_MOUSE | 25 | 0 | 0 | 1 | 19 |
| 198 | Syntaxin-8 OS = Mus musculus GN = Stx8 PE = 2 SV = 1 | STX8_MOUSE | 27 | 0 | 0 | 7 | 7 |
| 199 | Glycerol-3-phosphate dehydrogenase, mitochondrial OS = Mus musculus GN = Gpd2 PE = 1 SV = 2″ | GPDM_MOUSE | 81 | 0 | 0 | 8 | 7 |
| 200 | GTP-binding protein SAR1b OS = Cricetulus griseus GN = SAR1B PE = 1 SV = 1 | SAR1B_CRIGR (+2) | 22 | 0 | 0 | 1 | 14 |
| 205 | Prenylcysteine oxidase OS = Rattus norvegicus GN = Pcyox1 PE = 1 SV = 1 | PCYOX_RAT | 56 | 0 | 0 | 1 | 15 |
| 207 | Tetraspanin-3 OS = Mus musculus GN = Tspan3 PE = 1 SV = 1 | TSN3_MOUSE | 28 | 0 | 0 | 4 | 6 |
| 210 | Cytochrome c oxidase subunit 2 OS = Cavia aperea GN = MT-CO2 PE = 3 SV = 1 | COX2_CAVAP | 26 | 0 | 0 | 2 | 13 |
| 220 | Platelet glycoprotein 4 OS = Mesocricetus auratus GN = CD36 PE = 2 SV = 3 | CD36_MESAU | 53 | 0 | 0 | 1 | 8 |
| 223 | NADH-cytochrome b5 reductase 3 OS = Rattus norvegicus GN = Cyb5r3 PE = 1 SV = 2 | NB5R3_RAT | 34 | 0 | 0 | 6 | 8 |
| 224 | Mitochondrial import inner membrane translocase subunit TIM44 OS = Rattus norvegicus GN = Timm44 PE = 2 SV = 1 | TIM44_RAT | 51 | 0 | 0 | 2 | 11 |
| 231 | Ras-related protein Rab-31 OS = Mus musculus GN = Rab31 PE = 2 SV = 1 | RAB31_MOUSE (+1) | 21 | 0 | 0 | 3 | 12 |
| 234 | Lamina-associated polypeptide 2 isoforms beta/delta/epsilon/gamma OS = Mus musculus GN = Tmpo PE = 1 SV = 3 | LAP2B_MOUSE | 50 | 0 | 0 | 2 | 9 |
| 235 | Neutral amino acid transporter B(0) OS = Mus musculus GN = Slc1a5 PE = 2 SV = 2 | AAAT_MOUSE | 58 | 0 | 0 | 10 | 3 |
| 238 | Eukaryotic translation initiation factor 3 subunit A OS = Mus musculus GN = Eif3a PE = 2 SV = 3 | EIF3A_MOUSE | 162 | 0 | 0 | 11 | 1 |
| 239 | Acyl-coenzyme A thioesterase 1 OS = Rattus norvegicus GN = Acot1 PE = 1 SV = 1 | ACOT1_RAT | 46 | 0 | 0 | 11 | 2 |
| 240 | T-complex protein 1 subunit eta OS = Mus musculus GN = Cct7 PE = 1 SV = 1 | TCPH_MOUSE | 60 | 0 | 0 | 6 | 1 |
| 243 | Tumor protein D54 OS = Mus musculus GN = Tpd5212 PE = 1 SV = 1 | TPD54_MOUSE | 24 | 0 | 0 | 4 | 8 |
| 244 | Ras-related protein Rap-2b OS = Mus musculus GN = Rap2b PE = 1 SV = 1 | RAP2B_MOUSE (+1) | 21 | 0 | 0 | 10 | 3 |
| 247 | Basigin (Fragment) OS = Cricetulus griseus GN = BSG PE = 2 SV = 1 | BAS1_CRIGR | 27 | 0 | 0 | 1 | 13 |
| 248 | Tyrosine-protein phosphatase non-receptor type 1 OS = Rattus norvegicus GN = Ptpn1 PE = 2 SV = 1 | PTN1_RAT | 50 | 0 | 0 | 1 | 9 |
| 251 | Prostaglandin E synthase 2 OS = Mus musculus GN = Ptges2 PE = 1 SV = 2 | PGES2_MOUSE | 43 | 0 | 0 | 1 | 11 |
| 261 | Guanine nucleotide-binding protein G(s) subunit alpha isoforms XLas OS = Mus musculus GN = Gnas PE = 2 SV = 1 | GNAS1_MOUSE (+5) | 122 | 0 | 0 | 2 | 4 |
| 265 | Guanine nucleotide-binding protein G(I)/G(S)/G(T) subunit beta-2 OS = Mus musculus GN = Gnb2 PE = 1 SV = 3 | GBB2_MOUSE (+1) | 37 | 0 | 0 | 5 | 5 |
| 268 | Chronic lymphocytic leukemia deletion region gene 6 protein homolog OS = Mus musculus GN = Clld6 PE = 2 SV = 2 | CLLD6_MOUSE (+1) | 22 | 0 | 0 | 7 | 1 |
| 269 | Syntaxin-4 OS = Mus musculus GN = Stx4 PE = 1 SV = 1 | STX4_MOUSE | 34 | 0 | 0 | 2 | 10 |
| 270 | Lysosome-associated membrane glycoprotein 2 OS = Cricetulus griseus GN = LAMP2 PE = 2 SV = 1 | LAMP2_CRIGR | 45 | 0 | 0 | 1 | 11 |
| 272 | Mitochondrial carnitine/acylcarnitine carrier protein OS = Mus musculus GN = Slc25a20 PE = 1 SV = 1 | MCAT_MOUSE | 33 | 0 | 0 | 1 | 8 |
| 274 | Guanine nucleotide-binding protein alpha-11 subunit OS = Mus musculus GN = Gna11 PE = 1 SV = 1 | GNA11_MOUSE | 42 | 0 | 0 | 3 | 2 |
| 275 | Syntaxin-6 OS = Mus musculus GN = Stx6 PE = 2 SV = 1 | STX6_MOUSE (+1) | 29 | 0 | 0 | 4 | 9 |
| 276 | 40S ribosomal protein S11 OS = Mus musculus GN = Rps11 PE = 2 SV = 3 | RS11_MOUSE (+1) | 18 | 0 | 0 | 5 | 5 |
| 278 | Eukaryotic translation initiation factor 3 subunit C OS = Mus musculus GN = Eif3c PE = 1 SV = 1 | EIF3C_MOUSE | 106 | 0 | 0 | 7 | 0 |
| 281 | Heterogeneous nuclear ribonucleoprotein F OS = Mus musculus GN = Hnrnpf PE = 1 SV = 3 | HNRPF_MOUSE (+1) | 46 | 0 | 0 | 7 | 3 |
| 283 | Dolichyl-diphosphooligosaccharide--protein glycosyltransferase subunit STT3A OS = Mus musculus GN = Stt3a PE = 1 SV = 1 | STT3A_MOUSE | 81 | 0 | 0 | 1 | 4 |
| 285 | 60S ribosomal protein L18a OS = Mus musculus GN = Rp118a PE = 2 SV = 1 | RL18A_MOUSE (+1) | 21 | 0 | 0 | 2 | 4 |
| 289 | Synaptic vesicle membrane protein VAT-1 homolog OS = Mus musculus GN = Vat1 PE = 2 SV = 3 | VAT1_MOUSE | 43 | 0 | 0 | 3 | 8 |
| 290 | Protein BAT5 OS = Mus musculus GN = Bat5 PE = 1 SV = 3 | BAT5_MOUSE | 63 | 0 | 0 | 1 | 8 |
| 293 | Sideroflexin-3 OS = Mus musculus GN = Sfxn3 PE = 1 SV = 1 | SFXN3_MOUSE | 35 | 0 | 0 | 1 | 7 |
| 294 | GTP:AMP phosphotransferase mitochondrial OS = Mus musculus GN = Ak3 PE = 1 SV = 3 | KAD3_MOUSE | 25 | 0 | 0 | 7 | 2 |
| 299 | Phosphatidylinositol 4-kinase type 2-alpha OS = Mus musculus GN = Pi4k2a PE = 1 SV = 1 | P4K2A_MOUSE | 54 | 0 | 0 | 7 | 1 |
| 300 | Lysosomal alpha-glucosidase OS = Rattus norvegicus GN = Gaa PE = 2 SV = 1 | LYAG_RAT | 106 | 0 | 0 | 2 | 7 |
| 301 | Phytanoyl-CoA dioxygenase, peroxisomal OS = Mus musculus GN = Phyh PE = 1 SV = 1 | PAHX_MOUSE | 39 | 0 | 0 | 7 | 1 |
| 307 | NADH dehydrogenase [ubiquinone] iron-sulfur protein 7, mitochondrial OS = Mus musculus GN = Ndufs7 PE = 1 SV = 1 | NDUS7_MOUSE | 25 | 0 | 0 | 5 | 6 |
| 308 | Secretory carrier-associated membrane protein 2 OS = Mus musculus GN = Scamp2 PE = 2 SV = 1 | SCAM2_MOUSE | 36 | 0 | 0 | 2 | 2 |
| 309 | ATP synthase subunit gamma, mitochondrial OS = Rattus norvegicus GN = Atp5c1 PE = 1 SV = 2 | ATPG_RAT | 30 | 0 | 0 | 7 | 1 |
| 311 | 40S ribosomal protein S8 OS = Mus musculus GN = Rps8 PE = 1 SV = 2 | RS8_MOUSE (+1) | 24 | 0 | 0 | 6 | 4 |
| 313 | 60S ribosomal protein L10 OS = Mus musculus GN = Rpl10 PE = 2 SV = 3 | RL10_MOUSE (+1) | 25 | 0 | 0 | 2 | 3 |
| 315 | Mitochondrial glutamate carrier 1 OS = Mus musculus GN = Slc25a22 PE = 1 SV = 1 | GHC1_MOUSE | 35 | 0 | 0 | 1 | 4 |
| 316 | T-complex protein 1 subunit delta OS = Rattus norvegicus GN = Cct4 PE = 1 SV = 3 | TCPD_RAT | 58 | 0 | 0 | 4 | 2 |
| 317 | Transmembrane protein 85 OS = Mus musculus GN = Tmem85 PE = 2 SV = 1 | TMM85_MOUSE | 20 | 0 | 0 | 3 | 8 |
| 321 | 60S ribosomal protein L6 OS = Rattus norvegicus GN = Rpl6 PE = 1 SV = 5 | RL6_RAT | 34 | 0 | 0 | 2 | 5 |
| 322 | Nicastrin OS = Mus musculus GN = Ncstn PE = 1 SV = 2 | NICA_MOUSE | 78 | 0 | 0 | 2 | 7 |
| 328 | Coatomer subunit gamma OS = Mus musculus GN = Copg PE = 2 SV = 1 | COPG_MOUSE (+1) | 98 | 0 | 0 | 5 | 2 |
| 329 | 40S ribosomal protein S3a OS = Mus musculus GN = Rps3a PE = 1 SV = 3 | RS3A_MOUSE (+1) | 30 | 0 | 0 | 5 | 3 |
| 332 | Cytochrome b-c1 complex subunit 1, mitochondrial OS = Mus musculus GN = Uqcrc1 PE = 1 SV = 1 | QCR1_MOUSE (+1) | 53 | 0 | 0 | 5 | 2 |
| 333 | Protein disulfide-isomerase TXNDC10 OS = Mus musculus GN = Txndc10 PE = 1 SV = 2 | TXD10_MOUSE | 52 | 0 | 0 | 4 | 6 |
| 344 | Ras GTPase-activating protein-binding protein 1 OS = Mus musculus GN = G3bp1 PE = 1 SV = 1 | G3BP1_MOUSE | 52 | 0 | 0 | 3 | 2 |
| 350 | Myoferlin OS = Mus musculus GN = Fer113 PE = 2 SV = 2 | MYOF_MOUSE | 233 | 0 | 0 | 3 | 2 |
| 351 | NADH-cytochrome b5 reductase 1 OS = Rattus norvegicus GN = Cyb5r1 PE = 2 SV = 1 | NB5R1_RAT | 34 | 0 | 0 | 1 | 7 |
| 352 | Galectin-3 OS = Cricetulus longicaudatus GN = LGALS3 PE = 2 SV = 2 | LEG3_CRILO | 26 | 0 | 0 | 4 | 2 |
| 355 | Squalene synthetase OS = Mus musculus GN = Fdft1 PE = 2 SV = 1 | FDFT_MOUSE | 48 | 0 | 0 | 1 | 8 |
| 356 | NADH-ubiquinone oxidoreductase 75 subunit, mitochondrial OS = Mus musculus GN = Ndufs1 PE = 1 SV = 1 | NDUS1_MOUSE | 80 | 0 | 0 | 5 | 3 |
| 360 | Abhydrolase domain-containing protein 5 OS = Mus musculus GN = Abhd5 PE = 1 SV = 1 | ABHD5_MOUSE (+1) | 39 | 0 | 0 | 1 | 6 |
| 361 | Golgi apparatus protein 1 OS = Cricetulus griseus GN = GLG1 PE = 1 SV = 1 | GSLG1_CRIGR (+1) | 132 | 0 | 0 | 3 | 3 |
| 365 | ADP-ribosylation factor-like protein 6-interacting protein 1 OS = Mus musculus GN = Arl6ip1 PE = 2 SV = 1 | AR6P1_MOUSE | 23 | 0 | 0 | 1 | 5 |
| 369 | CDP-diacylglycerol--inositol 3-phosphatidyltransferase OS = Rattus norvegicus GN = Cdipt PE = 1 SV = 1 | CDIPT_RAT | 24 | 0 | 0 | 1 | 8 |
| 370 | Transmembrane emp24 domain-containing protein 9 OS = Mus musculus GN = Tmed9 PE = 2 SV = 1 | TMED9_MOUSE | 25 | 0 | 0 | 1 | 5 |
| 372 | Integrin alpha-3 OS = Cricetulus griseus GN = ITGA3 PE = 1 SV = 2 | ITA3_CRIGR | 119 | 0 | 0 | 1 | 5 |
| 375 | Peptidyl-tRNA hydrolase 2, mitochondrial OS = Mus musculus GN = Ptrh2 PE = 2 SV = 1 | PTH2_MOUSE | 20 | 0 | 0 | 1 | 2 |
| 379 | ADP-ribosylation factor 5 OS = Mus musculus GN = Arf5 PE = 2 SV = 2 | ARF5_MOUSE (+1) | 21 | 0 | 0 | 6 | 3 |
| 382 | Synaptobrevin homolog YKT6 OS = Mus musculus GN = Ykt6 PE = 2 SV = 1 | YKT6_MOUSE | 22 | 0 | 0 | 1 | 4 |
| 384 | Ras-related protein Ral-B OS = Rattus norvegicus GN = Ralb PE = 2 SV = 1 | RALB_RAT | 23 | 0 | 0 | 3 | 2 |
| 385 | Cleavage and polyadenylation specificity factor subunit 5 OS = Mus musculus GN = Nudt21 PE = 2 SV = 1 | CPSF5_MOUSE (+1) | 26 | 0 | 0 | 3 | 1 |
| 389 | AP-2 complex subunit alpha-1 OS = Mus musculus GN = Ap2a1 PE = 1 SV = 1 | AP2A1_MOUSE | 108 | 0 | 0 | 3 | 1 |
| 392 | GTPase HRas OS = Mus musculus GN = Hras1 PE = 1 SV = 1 | RASH_MOUSE (+1) | 21 | 0 | 0 | 4 | 3 |
| 393 | 40S ribosomal protein S2 OS = Mus musculus GN = Rps2 PE = 1 SV = 3 | RS2_MOUSE (+1) | 31 | 0 | 0 | 4 | 0 |
| 394 | Dihydroxyacetone phosphate acyltransferase OS = Mus musculus GN = Gnpat PE = 2 SV = 1 | GNPAT_MOUSE | 77 | 0 | 0 | 3 | 1 |
| 396 | FK506-binding protein 8 OS = Mus musculus GN = Fkbp8 PE = 1 SV = 2 | FKBP8_MOUSE (+1) | 44 | 0 | 0 | 1 | 4 |
| 397 | Ras-related protein Rab-5B OS = Mus musculus GN = Rab5b PE = 1 SV = 1 | RAB5B_MOUSE | 24 | 0 | 0 | 2 | 4 |
| 399 | Abhydrolase domain-containing protein FAM108B1 OS = Mus musculus GN = Fam108b1 PE = 2 SV = 1 | F108B_MOUSE (+1) | 32 | 0 | 0 | 4 | 1 |
| 400 | Eukaryotic translation initiation factor 3 subunit H OS = Mus musculus GN = Eif3h PE = 2 SV = 1 | EIF3H_MOUSE | 40 | 0 | 0 | 4 | 1 |
| 403 | Eukaryotic translation initiation factor 2 subunit I OS = Mus musculus GN = Eif2sI PE = 1 SV = 3 | IF2A_MOUSE (+1) | 36 | 0 | 0 | 4 | 0 |
| 405 | UPF0568 protein C14orf166 homolog OS = Mus musculus PE = 2 SV = 1 | CN166_MOUSE | 28 | 0 | 0 | 5 | 2 |
| 407 | Vesicle-associated membrane protein 7 OS = Mus musculus GN = Vamp7 PE = 2 SV = 1 | VAMP7_MOUSE | 25 | 0 | 0 | 7 | 0 |
| 411 | Alkyldihydroxyacetonephosphate synthase, peroxisomal OS = Mus musculus GN = Agps PE = 1 SV = 1 | ADAS_MOUSE | 72 | 0 | 0 | 4 | 1 |
| 412 | Thioredoxin domain-containing protein 1 OS = Mus musculus GN = Txndc1 PE = 1 SV = 1 | TXND1_MOUSE | 31 | 0 | 0 | 1 | 5 |
| 415 | 60S ribosomal protein L23a OS = Mus musculus GN = Rpl23a PE = 2 SV = 1 | RL23A_MOUSE (+1) | 18 | 0 | 0 | 4 | 2 |
| 416 | Golgi SNAP receptor complex member 2 OS = Rattus norvegicus GN = Gosr2 PE = 1 SV = 2 | GOSR2_RAT | 25 | 0 | 0 | 1 | 3 |
| 418 | Peroxisomal carnitine O-octanoyltransferase OS = Rattus norvegicus GN = Crot PE = 1 SV = 3 | OCTC_RAT | 70 | 0 | 0 | 3 | 0 |
| 419 | CDK5 regulatory subunit-associated protein 1-like 1 OS = Mus musculus GN = Cdkal1 PE = 2 SV = 1 | CDKAL_MOUSE | 65 | 0 | 0 | 1 | 3 |
| 422 | Trifunctional enzyme subunit beta, mitochondrial OS = Mus musculus GN = Hadhb PE = 1 SV = 1 | ECHB_MOUSE | 51 | 0 | 0 | 2 | 2 |
| 425 | T-complex protein 1 subunit beta OS = Mus musculus GN = Cct2 PE = 1 SV = 4 | TCPB_MOUSE (+1) | 57 | 0 | 0 | 4 | 0 |
| 428 | Probable ATP-dependent RNA helicase DDX17 OS = Mus musculus GN = Ddx17 PE = 2 SV = 1 | DDX17_MOUSE | 72 | 0 | 0 | 4 | 3 |
| 429 | Guanine nucleotide-binding protein alpha-13 subunit OS = Mus musculus GN = Gna13 PE = 1 SV = 1 | GNA13_MOUSE | 44 | 0 | 0 | 2 | 3 |
| 432 | Eukaryotic translation initiation factor 3 subunit E OS = Mus musculus GN = Eif3e PE = 1 SV = 1 | EIF3E_MOUSE (+1) | 52 | 0 | 0 | 5 | 1 |
| 437 | Flotillin-2 OS = Mus musculus GN = Flot2 PE = 1 SV = 1 | FLOT2_MOUSE (+1) | 42 | 0 | 0 | 6 | 0 |
| 438 | Proteasome subunit beta type-3 OS = Rattus norvegicus GN = Psmb3 PE = 1 SV = 1 | PSB3_RAT | 23 | 0 | 0 | 1 | 2 |
| 440 | Hydroxymethylglutaryl-CoA lyase, mitochondrial OS = Rattus norvegicus GN = Hmgcl PE = 2 SV = 1 | HMGCL_RAT | 34 | 0 | 0 | 2 | 3 |
| 442 | Signal recognition particle receptor subunit alpha OS = Mus musculus GN = Srpr PE = 2 SV = 1 | SRPR_MOUSE | 70 | 0 | 0 | 3 | 1 |
| 443 | Alpha-soluble NSF attachment protein OS = Mus musculus GN = Napa PE = 1 SV = 1 | SNAA_MOUSE (+1) | 33 | 0 | 0 | 2 | 0 |
| 447 | D-3-phosphoglycerate dehydrogenase OS = Mus musculus GN = Phgdh PE = 1 SV = 3 | SERA_MOUSE (+1) | 57 | 0 | 0 | 4 | 1 |
| 448 | 14-3-3 protein zeta/delta OS = Mus musculus GN = Ywhaz PE = 1 SV = 1 | 1433Z_MOUSE (+1) | 28 | 0 | 0 | 2 | 0 |
| 451 | 2-hydroxyacyl-CoA lyase 1 OS = Rattus norvegicus GN = Hacl1 PE = 1 SV = 1 | HACL1_RAT | 64 | 0 | 0 | 2 | 0 |
| 453 | Peptidyl-prolyl cis-trans isomerase C OS = Mus musculus GN = Ppic PE = 1 SV = 1 | PPIC_MOUSE | 23 | 0 | 0 | 5 | 0 |
| 456 | Interleukin enhancer-binding factor 2 OS = Mus musculus GN = Ilf2 PE = 1 SV = 1 | ILF2_MOUSE | 43 | 0 | 0 | 1 | 3 |
| 458 | Lamin-A/C OS = Mus musculus GN = Lmna PE = 1 SV = 2 | LMNA_MOUSE (+1) | 74 | 0 | 0 | 2 | 0 |
| 459 | Poly(U)-binding-splicing factor PUF60 OS = Mus musculus GN = Puf60 PE = 2 SV = 2 | PUF60_MOUSE (+1) | 60 | 0 | 0 | 5 | 1 |
| 461 | Chloride channel protein 6 OS = Mus musculus GN = Clcn6 PE = 2 SV = 1 | CLCN6_MOUSE | 97 | 0 | 0 | 4 | 0 |
| 467 | Eukaryotic initiation factor 4A-III OS = Mus musculus GN = Eif4a3 PE = 2 SV = 3 | IF4A3_MOUSE | 47 | 0 | 0 | 3 | 0 |
| 470 | Eukaryotic translation initiation factor 3 subunit B OS = Mus musculus GN = Eif3b PE = 1 SV = 1 | EIF3B_MOUSE (+1) | 91 | 0 | 0 | 2 | 0 |
| 473 | Acyl-CoA synthetase family member 2, mitochondrial OS = Mus musculus GN = Acsf2 PE = 2 SV = 1 | ACSF2_MOUSE | 68 | 0 | 0 | 3 | 0 |
| 474 | Inosine-5′-monophosphate dehydrogenase 2 OS = Mus musculus GN = lmpdh2 PE = 1 SV = 2 | IMDH2_MOUSE | 56 | 0 | 0 | 2 | 1 |
| 475 | Ribosome-binding protein 1 OS = Mus musculus GN = Rrbp1 PE = 2 SV = 2 | RRBP1_MOUSE | 173 | 0 | 0 | 2 | 0 |
| 476 | Low-density lipoprotein receptor OS = Cricetulus griseus GN = LDLR PE = 3 SV = 1 | LDLR_CRIGR | 95 | 0 | 0 | 4 | 0 |
| 478 | D-beta-hydroxybutyrate dehydrogenase, mitochondrial OS = Mus musculus GN = Bdhl PE = 1 SV = 1 | BDH_MOUSE (+1) | 38 | 0 | 0 | 2 | 2 |
| 482 | Coatomer subunit delta OS = Mus musculus GN = Arcn1 PE = 2 SV = 1 | COPD_MOUSE (+1) | 57 | 0 | 0 | 3 | 1 |
| 483 | Developmentally-regulated GTP-binding protein 1 OS = Mus musculus GN = Drg1 PE = 1 SV = 1 | DRG1_MOUSE | 41 | 0 | 0 | 2 | 0 |
| 484 | Putative pre-mRNA-splicing factor ATP-dependent RNA helicase DHX15 OS = Mus musculus GN = Dhx15 PE = 2 SV = 2 | DHX15_MOUSE | 91 | 0 | 0 | 2 | 2 |
| 486 | ADP-ribosylation factor 6 OS = Mus musculus GN = Arf6 PE = 1 SV = 2 | ARF6_MOUSE (+1) | 20 | 0 | 0 | 4 | 1 |
| 487 | Niemann-Pick C1 protein OS = Mus musculus GN = Npc1 PE = 2 SV = 1 | NPC1_MOUSE | 143 | 0 | 0 | 3 | 1 |
| 488 | Copper chaperone for superoxide dismutase OS = Rattus norvegicus GN = Ccs PE = 1 SV = 1 | CCS_RAT | 29 | 0 | 0 | 2 | 0 |
| 491 | Vesicle-fusing ATPase OS = Cricetulus griseus GN = NSF PE = 1 SV = 1 | NSF_CRIGR | 83 | 0 | 0 | 1 | 3 |
| 493 | Proteasome subunit beta type-1 OS = Mus musculus GN = Psmb1 PE = 1 SV = 1 | PSB1_MOUSE (+1) | 26 | 0 | 0 | 2 | 0 |
| 497 | Uncharacterized protein C19orf43 homolog OS = Mus musculus PE = 2 SV = 1 | CS043_MOUSE | 18 | 0 | 0 | 5 | 0 |
| 498 | Actin-related protein 2/3 complex subunit 4 OS = Mus musculus GN = Arpc4 PE = 1 SV = 3 | ARPC4_MOUSE | 20 | 0 | 0 | 4 | 0 |
| 501 | Proteasome subunit beta type-5 OS = Mus musculus GN = Psmb5 PE = 1 SV = 3 | PSB5_MOUSE | 29 | 0 | 0 | 5 | 0 |
| 502 | Myosin regulatory light chain MRLC2 OS = Mus musculus GN = Mylc2b PE = 1 SV = 2 | MRLC2_MOUSE (+2) | 20 | 0 | 0 | 5 | 0 |
| 505 | 60S ribosomal protein L17 OS = Mus musculus GN = Rpl17 PE = 2 SV = 3 | RL17 MOUSE (+1) | 21 | 0 | 0 | 3 | 0 |
| 506 | Ras-related protein Rab-9B OS = Mus musculus GN = Rab9b PE = 2 SV = 1 | RAB9B_MOUSE | 23 | 0 | 0 | 1 | 2 |
| 509 | Receptor expression-enhancing protein 5 OS = Mus musculus GN = Reep5 PE = 1 SV = 1 | REEP5_MOUSE | 21 | 0 | 0 | 1 | 2 |
| 511 | Protein ITFG3 OS = Rattus norvegicus GN = Itfg3 PE = 1 SV = 1 | ITFG3_RAT | 61 | 0 | 0 | 2 | 1 |
| 513 | UPF0404 protein C11orf59 homolog OS = Mus musculus PE = 1 SV = 1 | CK059_MOUSE (+1) | 18 | 0 | 0 | 2 | 1 |
| 514 | Coatomer subunit beta OS = Mus musculus GN = Copb1 PE = 1 SV = 1 | COPB_MOUSE (+1) | 107 | 0 | 0 | 2 | 1 |
| 515 | Cytoplasmic FMR1-interacting protein 1 OS = Mus musculus GN = Cyfip1 PE = 1 SV = 1 | CYFP1_MOUSE | 145 | 0 | 0 | 3 | 0 |
| 516 | FK506-binding protein 10 OS = Mus musculus GN = Fkbp10 PE = 1 SV = 1 | FKB10_MOUSE | 65 | 0 | 0 | 2 | 0 |
| 519 | Rho-related GTP-binding protein RhoB OS = Mus musculus GN = Rhob PE = 1 SV = 1 | RHOB_MOUSE (+1) | 22 | 0 | 0 | 2 | 1 |
| 520 | Cell division control protein 2 homolog OS = Mus musculus GN = Cdc2 PE = 1 SV = 3 | CDC2_MOUSE (+1) | 34 | 0 | 0 | 2 | 1 |
| 525 | Dihydrolipoyl dehydrogenase, mitochondrial OS = Cricetulus griseus GN = DLD PE = 2 SV = 1 | DLDH_CRIGR | 54 | 0 | 0 | 3 | 1 |
| 526 | Lamin-B1 OS = Mus musculus GN = Lmnb1 PE = 1 SV = 3 | LMNB1_MOUSE (+1) | 67 | 0 | 0 | 3 | 1 |
| 528 | Eukaryotic translation initiation factor 3 subunit E-interacting protein OS = Mus musculus GN = Eif3eip PE = 2 SV = 1 | IF3E1_MOUSE | 67 | 0 | 0 | 3 | 0 |
| 529 | Calpain-5 OS = Mus musculus GN = Capn5 PE = 2 SV = 1 | CAN5_MOUSE | 73 | 0 | 0 | 2 | 0 |
| 530 | RuvB-like 1 OS = Mus musculus GN = Ruvbl1 PE = 1 SV = 1 | RUVB1_MOUSE (+1) | 50 | 0 | 0 | 3 | 0 |
| 533 | Eukaryotic translation initiation factor 4H OS = Mus musculus GN = Eif4h PE = 1 SV = 3 | IF4H_MOUSE (+1) | 27 | 0 | 0 | 2 | 0 |
| 537 | Microtubule-associated protein RP/EB family member 1 OS = Mus musculus GN = Mapre1 PE = 1 SV = 3 | MARE1_MOUSE (+1) | 30 | 0 | 0 | 4 | 0 |
| 545 | F-actin-capping protein subunit beta OS = Mus musculus GN = Capzb PE = 1 SV = 3 | CAPZB_MOUSE (+1) | 31 | 0 | 0 | 3 | 0 |
| 547 | 60S ribosome subunit biogenesis protein NIP7 homolog OS = Rattus norvegicus GN = Nip7 PE = 2 SV = 1 | NIP7_RAT | 20 | 0 | 0 | 3 | 0 |
| 549 | MOSC domain-containing protein 2, mitochondrial OS = Mus musculus GN = Mosc2 PE = 1 SV = 1 | MOSC2_MOUSE | 38 | 0 | 0 | 2 | 1 |
| 550 | Probable ATP-dependent RNA helicase DDX56 OS = Mus musculus GN = Ddx56 PE = 2 SV = 1 | DDX56_MOUSE | 61 | 0 | 0 | 2 | 1 |
| 553 | Protein EFR3 homolog A OS = Mus musculus GN = Efr3a PE = 2 SV = 1 | EFR3A_MOUSE | 93 | 0 | 0 | 3 | 0 |
| 554 | Lupus La protein homolog OS = Mus musculus GN = Ssb PE = 2 SV = 1 | LA_MOUSE | 48 | 0 | 0 | 2 | 0 |
| 560 | Chloride intracellular channel protein 1 OS = Mus musculus GN = Clic1 PE = 1 SV = 3 | CLIC1_MOUSE | 27 | 0 | 0 | 3 | 0 |
| 561 | Charged multivesicular body protein 1b-2 OS = Mus musculus GN = Chmp1b2 PE = 2 SV = 2 | CH1B2_MOUSE | 22 | 0 | 0 | 3 | 0 |
| 564 | Vacuolar ATP synthase subunit B, brain isoform OS = Mus musculus GN = Atp6v1b2 PE = 1 SV = 1 | VATB2_MOUSE (+1) | 57 | 0 | 0 | 3 | 0 |
| 565 | 60S ribosomal protein L3 OS = Mus musculus GN = Rpl3 PE = 2 SV = 2 | RL3_MOUSE (+1) | 46 | 0 | 0 | 3 | 0 |
| 568 | 14-3-3 protein gamma OS = Mus musculus GN = Ywhag PE = 1 SV = 2 | 1433G_MOUSE (+1) | 28 | 0 | 0 | 2 | 0 |
| 570 | Lysyl-tRNA synthetase OS = Cricetulus griseus GN = KARS PE = 2 SV = 1 | SYK_CRIGR (+1) | 68 | 0 | 0 | 2 | 0 |
| 572 | SPRY domain-containing protein 4 OS = Rattus norvegicus GN = Spryd4 PE = 2 SV = 1 | SPRY4_RAT (+1) | 23 | 0 | 0 | 2 | 0 |
| 574 | Phenylalanyl-tRNA synthetase alpha chain OS = Mus musculus GN = Farsa PE = 2 SV = 1 | SYFA_MOUSE | 58 | 0 | 0 | 2 | 0 |
| 576 | 40S ribosomal protein S7 OS = Mus musculus GN = Rps7 PE = 2 SV = 1 | RS7_MOUSE (+1) | 22 | 0 | 0 | 2 | 0 |
| 577 | Paralemmin OS = Mus musculus GN = Palm PE = 1 SV = 1 | PALM_MOUSE | 42 | 0 | 0 | 2 | 0 |
| 578 | Peptidyl-prolyl cis-trans isomerase H OS = Mus musculus GN = Ppih PE = 2 SV = 1 | PPIH_MOUSE | 20 | 0 | 0 | 2 | 0 |
| 585 | Anthrax toxin receptor 2 OS = Mus musculus GN = Antxr2 PE = 2 SV = 1 | ANTR2_MOUSE | 53 | 0 | 0 | 2 | 0 |
| 587 | Cellular nucleic acid-binding protein OS = Mus musculus GN = Cnbp PE = 2 SV = 2 | CNBP_MOUSE (+1) | 20 | 0 | 0 | 2 | 0 |
| 588 | 40S ribosomal protein S23 OS = Chinchilla lanigera GN = RPS23 PE = 2 SV = 1 | RS23_CHILA (+2) | 16 | 0 | 0 | 2 | 0 |
| 590 | DAZ-associated protein 1 OS = Mus musculus GN = Dazap1 PE = 2 SV = 2 | DAZP1_MOUSE | 43 | 0 | 0 | 2 | 0 |
| 593 | Ubiquitin carboxyl-terminal hydrolase 10 OS = Mus musculus GN = Usp10 PE = 1 SV = 2 | UBP10_MOUSE | 87 | 0 | 0 | 2 | 0 |
| 594 | F-actin-capping protein subunit alpha-1 OS = Rattus norvegicus GN = Capza1 PE = 1 SV = 1 | CAZA1_RAT | 33 | 0 | 0 | 2 | 0 |
| 595 | THO complex subunit 1 OS = Mus musculus GN = Thoc1 PE = 2 SV = 1 | THOC1_MOUSE | 75 | 0 | 0 | 2 | 0 |
| 600 | Eukaryotic translation initiation factor 4E OS = Mus musculus GN = Eif4e PE = 1 SV = 1 | IF4E_MOUSE (+1) | 25 | 0 | 0 | 2 | 0 |
| 601 | U2-associated protein SR140 OS = Mus musculus GN = Sr140 PE = 2 SV = 2 | SR140_MOUSE | 118 | 0 | 0 | 2 | 0 |
| 604 | RNA-binding protein 8A OS = Mus musculus GN = Rbm8a PE = 2 SV = 2 | RBM8A_MOUSE | 20 | 0 | 0 | 2 | 0 |

TABLE 2
Spectral count data of high-confidence proteins measured by tandem LC-MS from C15pacFA-labeled and
UV-irradiated CHO-cells. Proteins exclusively found in lane IV (FIG. 2, Panel D).

| Nr | Protein | AccNr | kDa | I | II | III | IV |
|---|---|---|---|---|---|---|---|
| 98 | Sorting and assembly machinery component 50 homolog OS = Mus musculus GN = Samm50 PE = 1 SV = 1 | SAM50_MOUSE | 52 | 0 | 0 | 0 | 25 |
| 130 | Heme oxygenase 2 OS = Mus musculus GN = Hmox2 PE = 2 SV = 1 | HMOX2_MOUSE | 36 | 0 | 0 | 0 | 26 |
| 136 | Coiled-coil domain-containing protein 47 OS = Mus musculus GN = Ccdc47 PE = 2 SV = 2 | CC47_MOUSE (+1) | 56 | 0 | 0 | 0 | 22 |
| 144 | LETM1 and EF-hand domain-containing protein 1, mitochondrial OS = Mus musculus GN = Letm1 PE = 2 SV = 1 | LETM1_MOUSE (+1) | 83 | 0 | 0 | 0 | 15 |
| 149 | Mitochondrial inner membrane protein OS = Mus musculus GN = Immt PE = 1 SV = 1 | IMMT_MOUSE | 84 | 0 | 0 | 0 | 19 |
| 156 | Emerin OS = Rattus norvegicus GN = Emd PE = 2 SV = 1 | EMD_RAT | 30 | 0 | 0 | 0 | 25 |
| 168 | B-cell receptor-associated protein 31 OS = Mus musculus GN = Bcap31 PE = 1 SV = 3 | BAP31_MOUSE | 28 | 0 | 0 | 0 | 16 |
| 170 | Sphingosine-1-phosphate lyase 1 OS = Rattus norvegicus GN = Sgpl1 PE = 2 SV = 1 | SGPL1_RAT | 64 | 0 | 0 | 0 | 19 |
| 187 | Reticulon-4 OS = Rattus norvegicus GN = Rtn4 PE = 1 SV = 1 | RTN4_RAT | 126 | 0 | 0 | 0 | 14 |
| 190 | 3-hydroxy-3-methylglutaryl-coenzyme A reductase OS = Cricetulus griseus GN = HMGCR PE = 3 SV = 1 | HMDH_CRIGR | 97 | 0 | 0 | 0 | 15 |
| 191 | Phosphatidylinositol transfer protein beta isoform OS = Mus musculus GN = Pitpnb PE = 1 SV = 2 | PIPNB_MOUSE (+1) | 31 | 0 | 0 | 0 | 15 |
| 216 | Phosphatidylinositide phosphatase SAC1 OS = Rattus norvegicus GN = Sacml1 PE = 1 SV = 1 | SAC1_RAT | 67 | 0 | 0 | 0 | 11 |
| 221 | Torsin-1A (Fragment) OS = Cricetus cricetus GN = TOR1A PE = 2 SV = 1 | TOR1A_CRICR | 31 | 0 | 0 | 0 | 14 |
| 222 | Transmembrane emp24 domain-containing protein 2 (Fragment) OS = Cricetulus griseus GN = TMED2 PE = 1 SV = 1 | TMED2_CRIGR (+2) | 22 | 0 | 0 | 0 | 10 |
| 226 | CAAX prenyl protease 1 homolog OS = Mus musculus GN = Zmpste24 PE = 1 SV = 2 | FACE1_MOUSE | 55 | 0 | 0 | 0 | 10 |
| 228 | Protein sel-1 homolog 1 OS = Mesocricetus auratus GN = Se1l1 PE = 2 SV = 1 | SE1L1_MESAU | 89 | 0 | 0 | 0 | 15 |
| 230 | Alpha-mannosidase 2 OS = Mus musculus GN = Man2a1 PE = 1 SV = 1 | MA2A1_MOUSE | 132 | 0 | 0 | 0 | 12 |
| 232 | Heme oxygenase 1 OS = Rattus norvegicus GN = Hmox1 PE = 1 SV = 1 | HMOX1_RAT | 33 | 0 | 0 | 0 | 10 |
| 236 | Squalene monooxygenase OS = Mus musculus GN = Sqle PE = 2 SV = 1 | ERG1_MOUSE | 64 | 0 | 0 | 0 | 9 |
| 246 | Aspartyl/asparaginyl beta-hydroxylase OS = Mus musculus GN = Asph PE = 2 SV = 1 | ASPH_MOUSE | 83 | 0 | 0 | 0 | 13 |
| 254 | Abhydrolase domain-containing protein 6 OS = Mus musculus GN = Abhd6 PE = 2 SV = 1 | ABHD6_MOUSE (+1) | 38 | 0 | 0 | 0 | 10 |
| 256 | Vesicular integral-membrane protein VIP36 OS = Mus musculus GN = Lman2 PE = 2 SV = 1 | LMAN2_MOUSE | 40 | 0 | 0 | 0 | 13 |
| 257 | Stromal interaction molecule 1 OS = Mus musculus GN = Stim1 PE = 1 SV = 1 | STIM1_MOUSE | 78 | 0 | 0 | 0 | 9 |
| 258 | Nicalin OS = Mus musculus GN = Ncln PE = 2 SV = 2 | NCLN_MOUSE | 63 | 0 | 0 | 0 | 10 |
| 259 | Vesicle transport protein SEC20 OS = Mus musculus GN = Bnip1 PE = 2 SV = 1 | SEC20_MOUSE | 26 | 0 | 0 | 0 | 6 |
| 260 | Translocation protein SEC63 homolog OS = Mus musculus GN = Sec63 PE = 1 SV = 3 | SEC63_MOUSE | 88 | 0 | 0 | 0 | 13 |
| 262 | FK506-binding protein 11 OS = Mus musculus GN = Fkbpl1 PE = 2 SV = 1 | FKB11_MOUSE | 22 | 0 | 0 | 0 | 10 |
| 273 | Coatomer subunit zeta-1 OS = Mus musculus GN = Copz1 PE = 2 SV = 1 | COPZ1_MOUSE | 20 | 0 | 0 | 0 | 13 |
| 277 | UPF0480 protein C15orf24 homolog OS = Mus musculus GN = ORF3 PE = 2 SV = 1 | CO024_MOUSE | 26 | 0 | 0 | 0 | 7 |
| 284 | Mitochondrial import receptor subunit TOM40 homolog OS = Mus musculus GN = Tomm40 PE = 2 SV = 3 | TOM40_MOUSE | 38 | 0 | 0 | 0 | 13 |
| 287 | Tricarboxylate transport protein, mitochondrial OS = Rattus norvegicus GN = Slc25a1 PE = 1 SV = 1 | TXTP_RAT | 34 | 0 | 0 | 0 | 9 |
| 288 | Fatty aldehyde dehydrogenase OS = Mus musculus GN = Aldh3a2 PE = 2 SV = 1 | AL3A2_MOUSE | 54 | 0 | 0 | 0 | 2 |
| 291 | Serine palmitoyltransferase 1 OS = Cricetulus griseus GN = SPTLC1 PE = 2 SV = 1 | SPTC1_CRIGR | 53 | 0 | 0 | 0 | 9 |
| 295 | Transducin beta-like 2 protein OS = Mus musculus GN = Tbl2 PE = 2 SV = 1 | TBL2_MOUSE | 50 | 0 | 0 | 0 | 8 |
| 296 | GTP-binding protein SAR1a OS = Mus musculus GN = Sar1a PE = 2 SV = 1 | SAR1A_MOUSE | 22 | 0 | 0 | 0 | 11 |
| 304 | BRI3-binding protein OS = Mus musculus GN = Bri3bp PE = 2 SV = 1 | BRI3B_MOUSE | 28 | 0 | 0 | 0 | 8 |
| 312 | UBX domain-containing protein 2 OS = Mus musculus GN = Ubxd2 PE = 1 SV = 1 | UBXD2_MOUSE | 56 | 0 | 0 | 0 | 5 |
| 318 | Uncharacterized protein C17orf62 homolog OS = Mus musculus PE = 2 SV = 2 | CQ062_MOUSE (+1) | 21 | 0 | 0 | 0 | 8 |
| 319 | Transmembrane protein 43 OS = Rattus norvegicus GN = Tmem43 PE = 2 SV = 1 | TMM43_RAT | 45 | 0 | 0 | 0 | 9 |
| 320 | Long-chain-fatty-acid--CoA ligase 4 OS = Rattus norvegicus GN = Acsl4 PE = 2 SV = 1 | ACSL4_RAT | 74 | 0 | 0 | 0 | 8 |
| 326 | Transmembrane protein 214 OS = Rattus norvegicus GN = Tmem214 PE = 2 SV = 1 | TM214_RAT | 77 | 0 | 0 | 0 | 6 |
| 327 | Succinate dehydrogenase [ubiquinone] iron-sulfur subunit, mitochondrial OS = Mus musculus GN = Sdhb PE = 1 SV = 1 | DHSB_MOUSE | 32 | 0 | 0 | 0 | 5 |
| 330 | Sulfide:quinone oxidoreductase, mitochondrial OS = Mus musculus GN = Sqrdl PE = 2 SV = 2 | SQRD_MOUSE | 50 | 0 | 0 | 0 | 9 |
| 334 | Golgi SNAP receptor complex member 1 OS = Cricetulus griseus GN = GOSR1 PE = 2 SV = 1 | GOSR1_CRIGR (+1) | 29 | 0 | 0 | 0 | 6 |
| 335 | Ras-related protein Rab-6A OS = Mus musculus GN = Rab6a PE = 1 SV = 4 | RAB6A_MOUSE | 24 | 0 | 0 | 0 | 8 |
| 346 | Mitochondrial folate transporter/carrier OS = Mus musculus GN = Slc25a32 PE = 2 SV = 1 | MFTC_MOUSE | 35 | 0 | 0 | 0 | 6 |
| 348 | Transmembrane protein 199 OS = Mus musculus GN = Tmem199 PE = 2 SV = 1 | TM199_MOUSE | 23 | 0 | 0 | 0 | 3 |
| 353 | Calcium-binding mitochondrial carrier protein SCaMC-1 OS = Mus musculus GN = Slc25a24 PE = 2 SV = 1 | SCMC1_MOUSE | 53 | 0 | 0 | 0 | 5 |
| 354 | Inositol monophosphatase 3 OS = Mus musculus GN = Impad1 PE = 2 SV = 1 | IMPA3_MOUSE | 39 | 0 | 0 | 0 | 3 |
| 357 | Hexokinase-2 OS = Mus musculus GN = Hk2 PE = 1 SV = 1 | HXK2_MOUSE (+1) | 103 | 0 | 0 | 0 | 8 |
| 358 | Mitochondrial dicarboxylate carrier OS = Mus musculus GN = Slc25a10 PE = 2 SV = 2 | DIC_MOUSE | 32 | 0 | 0 | 0 | 7 |
| 366 | ADAM 10 OS = Mus musculus GN = Adam10 PE = 1 SV = 1 | ADA10_MOUSE | 84 | 0 | 0 | 0 | 6 |
| 367 | Matrix metalloproteinase-14 OS = Rattus norvegicus GN = Mmp14 PE = 2 SV = 2 | MMP14_RAT | 66 | 0 | 0 | 0 | 4 |
| 371 | Dehydrogenase/reductase SDR family member 1 OS = Mus musculus GN = Dhrs1 PE = 2 SV = 1 | DHRS1_MOUSE | 34 | 0 | 0 | 0 | 8 |
| 374 | Ras-related protein Rab-10 OS = Mus musculus GN = Rab10 PE = 1 SV = 1 | RAB10_MOUSE | 23 | 0 | 0 | 0 | 7 |
| 376 | Sigma I-type opioid receptor OS = Rattus norvegicus GN = Oprs1 PE = 1 SV = 1 | OPRS1_RAT | 25 | 0 | 0 | 0 | 4 |
| 377 | UBX domain-containing protein 8 OS = Mus musculus GN = Ubxd8 PE = 2 SV = 2 | UBXD8_MOUSE | 52 | 0 | 0 | 0 | 6 |
| 380 | Probable saccharopine dehydrogenase OS = Mus musculus GN = Sccpdh PE = 2 SV = 1 | SCPDH_MOUSE | 47 | 0 | 0 | 0 | 4 |
| 381 | Estradiol 17-beta-dehydrogenase 12 OS = Rattus norvegicus GN = Hsd17b12 PE = 2 SV = 1 | DHB12_RAT | 35 | 0 | 0 | 0 | 3 |
| 383 | Calcium-binding mitochondrial carrier protein Aralar1 OS = Mus musculus GN = Slc25a12 PE = 1 SV = 1 | CMC1_MOUSE | 75 | 0 | 0 | 0 | 7 |
| 388 | Amyloid beta A4 protein OS = Rattus norvegicus GN = App PE = 1 SV = 2 | A4_RAT | 87 | 0 | 0 | 0 | 6 |
| 390 | Vitamin K epoxide reductase complex subunit 1-like protein 1 OS = Mus musculus GN = VkorclII PE = 2 SV = 1 | VKORL_MOUSE (+1) | 20 | 0 | 0 | 0 | 6 |
| 395 | Atlastin-2 OS = Mus musculus GN = Arl6ip2 PE = 2 SV = 1 | ATLA2_MOUSE | 66 | 0 | 0 | 0 | 5 |
| 398 | Calnexin OS = Mus musculus GN = Canx PE = 1 SV = 1 | CALX_MOUSE | 67 | 0 | 0 | 0 | 7 |
| 401 | Suppressor of tumorigenicity protein 7 OS = Cavia porcellus GN = ST7 PE = 3 SV = 1 | ST7_CAVPO (+2) | 67 | 0 | 0 | 0 | 2 |
| 404 | Thioredoxin domain-containing protein 14 OS = Rattus norvegicus GN = Txndc14 PE = 2 SV = 1 | TXD14_RAT | 34 | 0 | 0 | 0 | 4 |
| 406 | AFG3-like protein 2 OS = Mus musculus GN = Afg312 PE = 1 SV = 1 | AFG32_MOUSE | 90 | 0 | 0 | 0 | 7 |
| 413 | Cleft lip and palate transmembrane protein I homolog OS = Mus musculus GN = Clptm1 PE = 1 SV = 1 | CLPT1_MOUSE | 75 | 0 | 0 | 0 | 6 |
| 414 | Neuroplastin OS = Mus musculus GN = Nptn PE = 1 SV = 2 | NPTN_MOUSE (+1) | 31 | 0 | 0 | 0 | 6 |
| 417 | Magnesium transporter protein 1 OS = Rattus norvegicus GN = Magt1 PE = 2 SV = 2 | MAGT1_RAT | 38 | 0 | 0 | 0 | 5 |
| 420 | UPF0510 protein C19orf63 homolog OS = Rattus norvegicus PE = 2 SV = 1 | CS063_RAT | 27 | 0 | 0 | 0 | 3 |
| 421 | Emerin OS = Mus musculus GN = Emd PE = 2 SV = 1 | EMD_MOUSE | 29 | 0 | 0 | 0 | 6 |
| 426 | Syntaxin-5 OS = Mus musculus GN = Stx5 PE = 2 SV = 3 | STX5_MOUSE (+1) | 40 | 0 | 0 | 0 | 4 |
| 427 | Derlin-1 OS = Mus musculus GN = Derl1 PE = 2 SV = 1 | DERL1_MOUSE | 29 | 0 | 0 | 0 | 4 |
| 433 | Fibronectin type III domain-containing protein 3B OS = Mus musculus GN = Fndc3b PE = 1 SV = 1 | FND3B_MOUSE | 133 | 0 | 0 | 0 | 4 |
| 434 | Armadillo repeat-containing X-linked protein 3 OS = Mus musculus GN = Armcx3 PE = 1 SV = 1 | ARMX3_MOUSE (+1) | 43 | 0 | 0 | 0 | 5 |
| 435 | Transmembrane emp24 domain-containing protein 5 OS = Mus musculus GN = Tmed5 PE = 2 SV = 1 | TMED5_MOUSE (+1) | 26 | 0 | 0 | 0 | 5 |
| 436 | ATP-binding cassette sub-family D member 3 OS = Rattus norvegicus GN = Abcd3 PE = 1 SV = 3 | ABCD3_RAT | 75 | 0 | 0 | 0 | 4 |
| 441 | Phosphatidylserine decarboxylase proenzyme OS = Cricetulus griseus GN = PISD PE = 1 SV = 2 | PISD_CRIGR (+1) | 47 | 0 | 0 | 0 | 4 |
| 444 | Protein FAM114A2 OS = Mus musculus GN = Fam114a2 PE = 1 SV = 2 | F1142_MOUSE | 54 | 0 | 0 | 0 | 5 |
| 449 | Dephospho-CoA kinase domain containing protein OS = Rattus norvegicus GN = Dcakd PE = 2 SV = 1 | DCAKD_RAT | 27 | 0 | 0 | 0 | 2 |
| 450 | Retinol dehydrogenase 11 OS = Mus musculus GN = Rdh11 PE = 2 SV = 2 | RDH11_MOUSE | 35 | 0 | 0 | 0 | 3 |
| 452 | Arsenical pump-driving ATPase OS = Mus musculus GN = Asna1 PE = 1 SV = 2 | ARSA1_MOUSE | 39 | 0 | 0 | 0 | 5 |
| 455 | Neuropathy target esterase OS = Mus musculus GN = Pnpla6 PE = 2 SV = 2 | PLPL6_MOUSE | 150 | 0 | 0 | 0 | 5 |
| 460 | rRNA 2′-O-methyltransferase fibrillarin OS = Mus musculus GN = Fbl PE = 2 SV = 2 | FBRL_MOUSE (+1) | 34 | 0 | 0 | 0 | 3 |
| 462 | Calcium signal-modulating cyclophilin ligand OS = Mus musculus GN = Camlg PE = 1 SV = 2 | CAMLG_MOUSE | 33 | 0 | 0 | 0 | 3 |
| 463 | Nucleoporin NUP53 OS = Rattus norvegicus GN = Nup35 PE = 2 SV = 1 | NUP53_RAT | 35 | 0 | 0 | 0 | 6 |
| 464 | NADH dehydrogenase [ubiquinone] 1 alpha subcomplex subunit 9, mitochondrial OS = Mus musculus GN = Ndufa9 PE = 1 SV = 1 | NDUA9_MOUSE (+1) | 43 | 0 | 0 | 0 | 6 |
| 465 | Cytochrome b5 OS = Mus musculus GN = Cyb5a PE = 1 SV = 2 | CYB5_MOUSE (+1) | 15 | 0 | 0 | 0 | 4 |
| 466 | Metaxin-1 OS = Mus musculus GN = Mtx1 PE = 1 SV = 1 | MTX1_MOUSE | 36 | 0 | 0 | 0 | 5 |
| 468 | Arylacetamide deacetylase-like 1 OS = Mus musculus GN = Aadacl1 PE = 2 SV = 1 | ADCL1_MOUSE | 46 | 0 | 0 | 0 | 5 |
| 471 | Transmembrane emp24 domain-containing protein 3 OS = Mus musculus GN = Tmed3 PE = 2 SV = 1 | TMED3_MOUSE (+1) | 25 | 0 | 0 | 0 | 5 |
| 472 | Renin receptor OS = Mus musculus GN = Atp6ap2 PE = 2 SV = 2 | RENR_MOUSE | 39 | 0 | 0 | 0 | 3 |
| 479 | GPI transamidase component PIG-S OS = Mus musculus GN = Pigs PE = 2 SV = 3 | PIGS_MOUSE (+1) | 62 | 0 | 0 | 0 | 4 |
| 481 | Endoplasmic reticulum-Golgi intermediate compartment protein 1 OS = Mus musculus GN = Ergic1 PE = 1 SV = 1 | ERGI1_MOUSE | 33 | 0 | 0 | 0 | 3 |
| 485 | Protein phosphatase 1L OS = Mus musculus GN = Ppm1l PE = 1 SV = 1 | PPM1L_MOUSE | 41 | 0 | 0 | 0 | 3 |
| 489 | Ubiquitin-conjugating enzyme E2 J1 OS = Mus musculus GN = Ube2j1 PE = 1 SV = 2 | UB2J1_MOUSE | 35 | 0 | 0 | 0 | 5 |
| 490 | Dolichyl-diphosphooligosaccharide--protein glycosyltransferase subunit 2 OS = Mus musculus GN = Rpn2 PE = 2 SV = 1 | RPN2_MOUSE (+1) | 69 | 0 | 0 | 0 | 3 |
| 492 | Transmembrane protein 209 OS = Mus musculus GN = Tmem209 PE = 2 SV = 1 | TM209_MOUSE | 63 | 0 | 0 | 0 | 2 |
| 494 | Vesicle-associated membrane protein 8 OS = Rattus norvegicus GN = Vamp8 PE = 1 SV = 1 | VAMP8_RAT | 11 | 0 | 0 | 0 | 4 |
| 495 | Protein unc-84 homolog B OS = Mus musculus GN = Unc84b PE = 1 SV = 2 | UN84B_MOUSE | 78 | 0 | 0 | 0 | 2 |
| 496 | Dihydroorotate dehydrogenase, mitochondrial OS = Rattus norvegicus GN = Dhodh PE = 1 SV = 1 | PYRD_RAT | 43 | 0 | 0 | 0 | 2 |
| 499 | Abhydrolase domain-containing protein 12 OS = Mus musculus GN = Abhd12 PE = 2 SV = 2 | ABD12_MOUSE | 45 | 0 | 0 | 0 | 4 |
| 500 | Golgin subfamily A member 5 OS = Mus musculus GN = Golga5 PE = 1 SV = 2 | GOGA5_MOUSE (+1) | 82 | 0 | 0 | 0 | 3 |
| 503 | Protein tyrosine phosphatase type IVA 2 OS = Mus musculus GN = Ptp4a2 PE = 1 SV = 1 | TP4A2_MOUSE (+3) | 19 | 0 | 0 | 0 | 3 |
| 504 | Syntaxin-18 OS = Mus musculus GN = Stx18 PE = 2 SV = 2 | STX18_MOUSE (+1) | 38 | 0 | 0 | 0 | 2 |
| 507 | Coiled-coil-helix-coiled-coil-helix domain-containing protein 6 OS = Mus musculus GN = Chchd6 PE = 2 SV = 1 | CHCH6_MOUSE | 30 | 0 | 0 | 0 | 3 |
| 512 | Palmitoyl-protein thioesterase 1 OS = Mus musculus GN = Ppt1 PE = 2 SV = 2 | PPT1_MOUSE | 34 | 0 | 0 | 0 | 4 |
| 518 | Polypeptide N-acetylgalactosaminyltransferase 1 OS = Rattus norvegicus GN = Galnt1 PE = 1 SV = 1 | GALT1_RAT | 64 | 0 | 0 | 0 | 2 |
| 521 | Uncharacterized protein KIAA0090 OS = Mus musculus GN = Kiaa0090 PE = 2 SV = 1 | K0090_MOUSE | 112 | 0 | 0 | 0 | 4 |
| 523 | Metaxin-2 OS = Mus musculus GN = Mtx2 PE = 1 SV = 1 | MTX2_MOUSE | 30 | 0 | 0 | 0 | 3 |
| 524 | Mitochondrial import inner membrane translocase subunit TIM50 OS = Mus musculus GN = Timm50 PE = 1 SV = 1 | TIM50_MOUSE | 40 | 0 | 0 | 0 | 4 |
| 527 | Sulfhydryl oxidase 2 OS = Mus musculus GN = Qsox2 PE = 2 SV = 1 | QSOX2_MOUSE | 78 | 0 | 0 | 0 | 3 |
| 531 | Dolichyl-phosphate beta-glucosyltransferase OS = Mus musculus GN = Alg5 PE = 2 SV = 1 | ALG5_MOUSE | 37 | 0 | 0 | 0 | 2 |
| 534 | Protein ERGIC-53 OS = Mus musculus GN = Lman1 PE = 2 SV = 1 | LMAN1_MOUSE (+1) | 58 | 0 | 0 | 0 | 2 |
| 535 | Transmembrane emp24 domain-containing protein 4 OS = Mus musculus GN = Tmed4 PE = 2 SV = 1 | TMED4_MOUSE | 26 | 0 | 0 | 0 | 3 |
| 536 | StAR-related lipid transfer protein 7 OS = Mus musculus GN = Stard7 PE = 2 SV = 1 | STAR7_MOUSE | 34 | 0 | 0 | 0 | 4 |
| 538 | Transmembrane protein 65 OS = Mus musculus GN = Tmem65 PE = 2 SV = 1 | TMM65_MOUSE | 25 | 0 | 0 | 0 | 4 |
| 539 | Oxidoreductase HTATIP2 OS = Mus musculus GN = Htatip2 PE = 1 SV = 2 | HTAI2_MOUSE | 27 | 0 | 0 | 0 | 3 |
| 540 | AFG3-like protein 1 OS = Mus musculus GN = Afg3l1 PE = 2 SV = 2 | AFG31_MOUSE | 87 | 0 | 0 | 0 | 2 |
| 541 | Hydroxysteroid dehydrogenase-like protein 1 OS = Rattus norvegicus GN = Hsdl1 PE = 2 SV = 1 | HSDL1_RAT (+1) | 37 | 0 | 0 | 0 | 3 |
| 542 | Peroxisomal membrane protein 11B OS = Mus musculus GN = Pex11b PE = 2 SV = 1 | PX11B_MOUSE | 29 | 0 | 0 | 0 | 3 |
| 543 | Transmembrane protein 214 OS = Mus musculus GN = Tmem214 PE = 2 SV = 1 | TM214_MOUSE | 76 | 0 | 0 | 0 | 2 |
| 544 | Motile sperm domain-containing protein 2 OS = Mus musculus GN = Mospd2 PE = 1 SV = 2 | MSPD2_MOUSE | 60 | 0 | 0 | 0 | 2 |
| 548 | Apolipoprotein O OS = Mus musculus GN = Apoo PE = 2 SV = 1 | APOO_MOUSE | 24 | 0 | 0 | 0 | 3 |
| 551 | Protein Noxp20 OS = Mus musculus GN = Fam114a1 PE = 2 SV = 1 | NXP20_MOUSE | 61 | 0 | 0 | 0 | 3 |
| 555 | Cysteine-rich with EGF-like domain protein 1 OS = Mus musculus GN = Creld1 PE = 2 SV = 1 | CREL1_MOUSE (+1) | 46 | 0 | 0 | 0 | 2 |
| 556 | Vitamin K-dependent gamma-carboxylase OS = Mus musculus GN = Ggcx PE = 2 SV = 1 | VKGC_MOUSE | 87 | 0 | 0 | 0 | 2 |
| 557 | LEM domain-containing protein 2 OS = Mus musculus GN = Lemd2 PE = 1 SV = 1 | LEMD2_MOUSE | 58 | 0 | 0 | 0 | 2 |
| 558 | Fatty acyl-CoA reductase 1 OS = Mus musculus GN = Far1 PE = 1 SV = 1 | FACR1_MOUSE | 59 | 0 | 0 | 0 | 3 |
| 559 | Ras-related protein Rab-24 OS = Mus musculus GN = Rab24 PE = 2 SV = 2 | RAB24_MOUSE | 23 | 0 | 0 | 0 | 3 |
| 562 | Synaptojanin-2-binding protein OS = Rattus norvegicus GN = Synj2bp PE = 2 SV = 1 | SYJ2B_RAT | 23 | 0 | 0 | 0 | 3 |
| 563 | GPI-anchor transamidase OS = Mus musculus GN = Pigk PE = 2 SV = 2 | GPI8_MOUSE | 45 | 0 | 0 | 0 | 3 |
| 566 | Uncharacterized protein C20orf116 homolog OS = Mus musculus PE = 1 SV = 2 | CT116_MOUSE | 36 | 0 | 0 | 0 | 2 |
| 567 | UPF0420 protein C16orf58 homolog OS = Mus musculus PE = 2 SV = 1 | CP058_MOUSE | 50 | 0 | 0 | 0 | 2 |
| 569 | Signal recognition particle 68 protein OS = Mus musculus GN = Srp68 PE = 2 SV = 1 | SRP68_MOUSE | 71 | 0 | 0 | 0 | 2 |
| 571 | Mitochondrial 2-oxoglutarate/malate carrier protein OS = Mus musculus GN = Slc25a11 PE = 1 SV = 3 | M2OM_MOUSE | 34 | 0 | 0 | 0 | 2 |
| 573 | NADH dehydrogenase [ubiquinone] 1 alpha subcomplex subunit 10, mitochondrial OS = Mus musculus GN = Ndufa10 PE = 1 SV = 1 | NDUAA_MOUSE | 41 | 0 | 0 | 0 | 2 |
| 575 | Syntaxin-7 OS = Mus musculus GN = Stx7 PE = 1 SV = 3 | STX7_MOUSE | 30 | 0 | 0 | 0 | 2 |
| 580 | Ras-related protein Rab-32 OS = Mus musculus GN = Rab32 PE = 2 SV = 3 | RAB32_MOUSE | 25 | 0 | 0 | 0 | 2 |
| 581 | Sorting nexin-14 OS = Mus musculus GN = Snx14 PE = 2 SV = 1 | SNX14_MOUSE | 109 | 0 | 0 | 0 | 2 |
| 582 | Soluble calcium-activated nucleotidase 1 OS = Mus musculus GN = Cant1 PE = 2 SV = 1 | CANT1_MOUSE | 46 | 0 | 0 | 0 | 2 |
| 583 | Ras-related protein Rab-21 OS = Mus musculus GN = Rab21 PE = 1 SV = 4 | RAB21_MOUSE (+1) | 24 | 0 | 0 | 0 | 3 |
| 584 | Squalene synthetase OS = Rattus norvegicus GN = Fdft1 PE = 2 SV = 1 | FDFT_RAT | 48 | 0 | 0 | 0 | 2 |
| 586 | Protein jagunal homolog 1 OS = Mus musculus GN = Jagn1 PE = 2 SV = 2 | JAGN1_MOUSE (+1) | 21 | 0 | 0 | 0 | 3 |
| 589 | Interleukin-6 receptor subunit beta OS = Rattus norvegicus GN = I16st PE = 2 SV = 1 | IL6RB_RAT | 102 | 0 | 0 | 0 | 2 |
| 591 | E3 ubiquitin-protein ligase MARCH5 OS = Mus musculus GN = March5 PE = 2 SV = 1 | MARH5_MOUSE | 31 | 0 | 0 | 0 | 2 |
| 592 | Cytochrome c oxidase subunit 4 isoform 1, mitochondrial OS = Mus musculus GN = Cox4i1 PE = 1 SV = 2 | COX41_MOUSE | 20 | 0 | 0 | 0 | 2 |
| 596 | Cytochrome c-type heme lyase OS = Mus musculus GN = Hccs PE = 2 SV = 1 | CCHL_MOUSE | 31 | 0 | 0 | 0 | 2 |
| 597 | Long-chain specific acyl-CoA dehydrogenase, mitochondrial OS = Mus musculus GN = Acadl PE = 2 SV = 2 | ACADL_MOUSE | 48 | 0 | 0 | 0 | 2 |
| 598 | Transmembrane protein 111 OS = Mus musculus GN = Tmem111 PE = 2 SV = 3 | TM111_MOUSE (+1) | 30 | 0 | 0 | 0 | 2 |
| 599 | Neudesin OS = Rattus norvegicus GN = Nenf PE = 2 SV = 1 | NENF_RAT | 19 | 0 | 0 | 0 | 2 |
| 602 | Nurim OS = Mus musculus GN = Nrm PE = 2 SV = 1 | NRM_MOUSE (+1) | 29 | 0 | 0 | 0 | 2 |
| 603 | Ras-related protein Rab-4A OS = Mus musculus GN = Rab4a PE = 1 SV = 1 | RAB4A_MOUSE (+1) | 24 | 0 | 0 | 0 | 2 |

TABLE 3
Spectral count data of a subset of high-confidence proteins as listed in Table 2 with a cytosolic
localization. For complete AccNr., see Table 2. Proteins that are not lipid-modified and lack a
transmembrane domain are highlighted in red. Roman numbers refer to the lane of the Coomassie
stained gel in FIG. 2, Panel D.

| Nr | Protein | AccNr | kDa | I | II | III | IV |
|---|---|---|---|---|---|---|---|
| 98 | Sorting and assembly machinery component 50 | SAM50_MOUSE | 52 | 0 | 0 | 0 | 25 |
| | homolog | | | | | | |
| 191 | Phosphatidylinositol transfer protein beta isoform | PIPNB_MOUSE | 31 | 0 | 0 | 0 | 15 |
| 222 | Transmembrane emp24 domain-containing protein 2 | TMED2_CRIGR | 22 | 0 | 0 | 0 | 10 |
| 273 | Coatomer subunit zeta-1 | COPZ1_MOUSE | 20 | 0 | 0 | 0 | 13 |
| 377 | UBX domain-containing protein 8 | UBXD8_MOUSE | 52 | 0 | 0 | 0 | 6 |
| 452 | Arsenical pump-driving ATPase | ARSA1_MOUSE | 39 | 0 | 0 | 0 | 5 |
| 455 | Neuropathy target esterase | PLPL6_MOUSE | 150 | 0 | 0 | 0 | 5 |
| 503 | Protein tyrosine phosphatase type IVA 2 | TP4A2_MOUSE | 19 | 0 | 0 | 0 | 3 |
| 539 | Oxidoreductase HTATIP2 | HTAI2_MOUSE | 27 | 0 | 0 | 0 | 3 |
| 551 | Protein Noxp20 | NXP20_MOUSE | 61 | 0 | 0 | 0 | 3 |
| 559 | Ras-related protein Rab-24 | RAB24_MOUSE | 23 | 0 | 0 | 0 | 3 |
| 569 | Signal recognition particle 68 protein | SRP68_MOUSE | 71 | 0 | 0 | 0 | 2 |
| 583 | Ras-related protein Rab-21 | RAB21_MOUSE | 24 | 0 | 0 | 0 | 3 |
| 603 | Ras-related protein Rab-4A | RAB4A_MOUSE | 24 | 0 | 0 | 0 | 2 |

| Nr | Cellular component | PTM |
|---|---|---|
| 98 | Cytoplasm; Membrane; Mitochondrion | — |
| 191 | Cytoplasm; Golgi apparatus | Acetylation; Phosphoprotein |
| 222 | Cytoplasmic vesicle; Golgi apparatus; Membrane | — |
| 273 | Cytoplasm; Cytoplasmic vesicle; Golgi apparatus; Membrane | Acetylation; Phosphoprotein |
| 377 | Cytoplasm; Endoplasmic reticulum; Lipid droplet | Acetylation; Phosphoprotein |
| 452 | Cytoplasm; Endoplasmic reticulum; Nucleus | Acetylation |
| 455 | Endoplasmic reticulum Membrane | Phosphoprotein; Glycoprotein |
| 503 | Cell membrane; Cytoplasm; Endosome; Membrane | Lipoprotein, Prenylation, |
| | | Disulfide bond |
| 539 | Cytoplasm; Nucleus | Phosphoprotein |
| 551 | Cytoplasm | Phosphoprotein |
| 559 | Cytoplasm; Membrane | Lipoprotein; Prenylation |
| 569 | Cytoplasm; Nucleus; Signal recognition particle | Acetylation; Phosphoprotein |
| 583 | Cytoplasmic vesicle; Endoplasmic reticulum; Endosome; Golgi | Acetylation; Lipoprotein; |
| | appartus; Membrane | Prenylation; Methylation |
| 603 | Cytoplasm; Membrane | Lipoprotein; Prenylation; |
| | | Methylation; Phosphoprotein |

TABLE 4
Spectral count data of high-confidence ceramide interacting proteins from pac-lipid labeled cytosol fractions isolated
from GM95 cells. Displayed are data from two independent experiments with experiment #1 showing spectral counts for samples that were
treated with and without UV-irradiation. Experiment #2 shows spectral counts for samples that were treated with and without UV-irradiation in
the presence and absence of the indicated pac-lipid. Highlighted is the ceramide transfer protein CERT (Collagen type IV alpha-3-binding
protein). The following criteria were applied to define predicted ceramide-binding proteins that appeared in either of the two experiments:
(i) no spectral counts in the control (ctr I, II, III) of both experiments with ≧3 spectral counts in the C15pacCer labeled samples; (ii) spectral
counts of ≧10 for proteins found after cross-linking applying C15pacCer and with ≦10 spectra in the controls (ctr I, II, III). A second criteria
was applied with a spectral count ratio of ≧8. In order to take hits into account without any spectra in the controls, the number of spectra was
increased by 1 and the ratio was determined using the average of the spectra in the controls. (iii) For (i) and (ii), a second criteria was introduced:
spectral count ratios of C15pacCer were divided by spectral count ratios of C15pacGlcCer and proteins with a ration of ≧1 were taken into account.

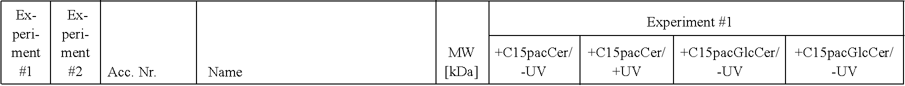

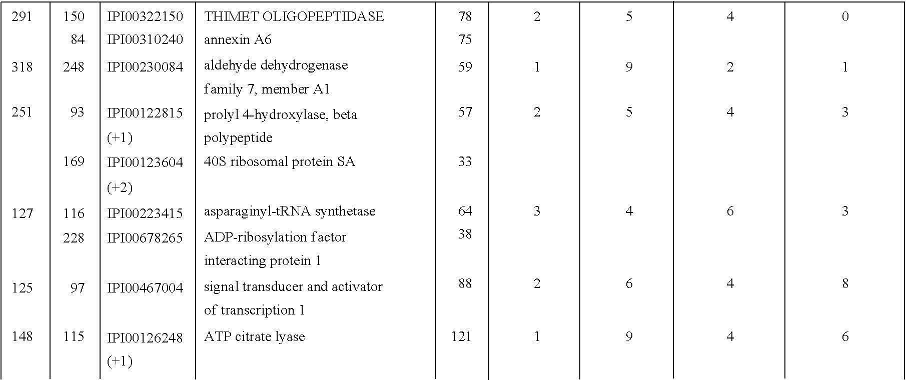

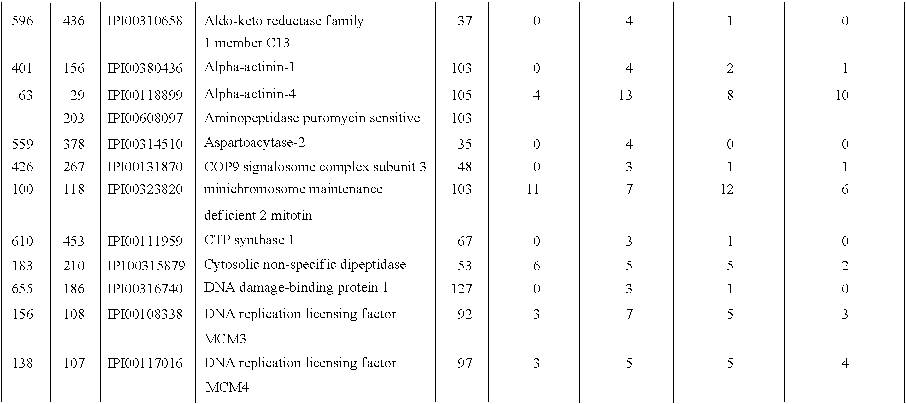

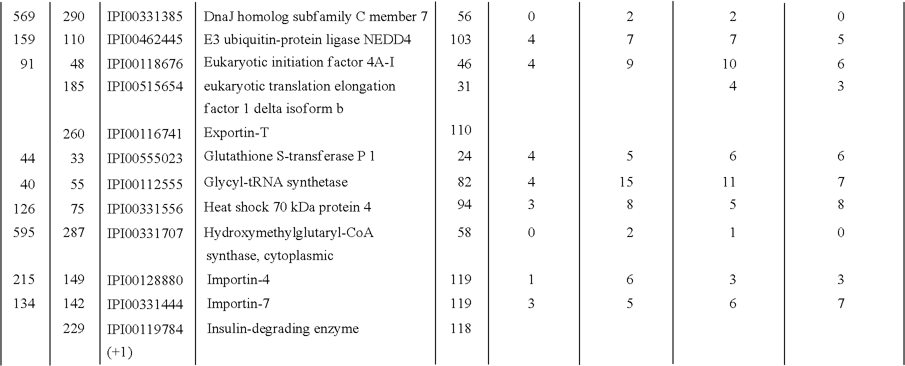

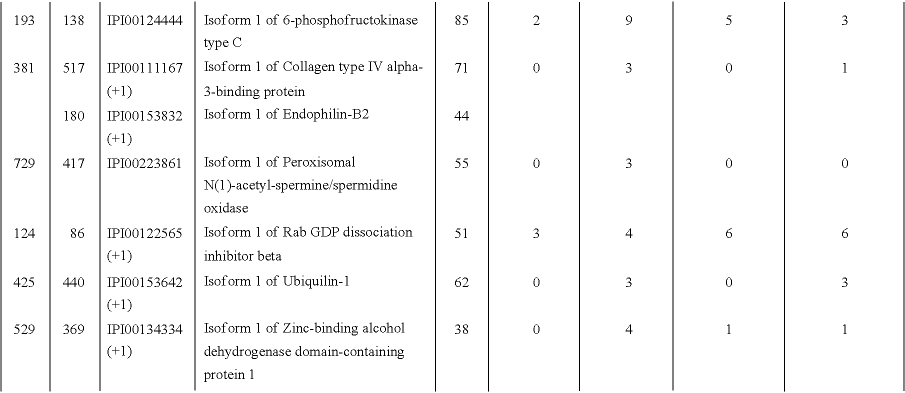

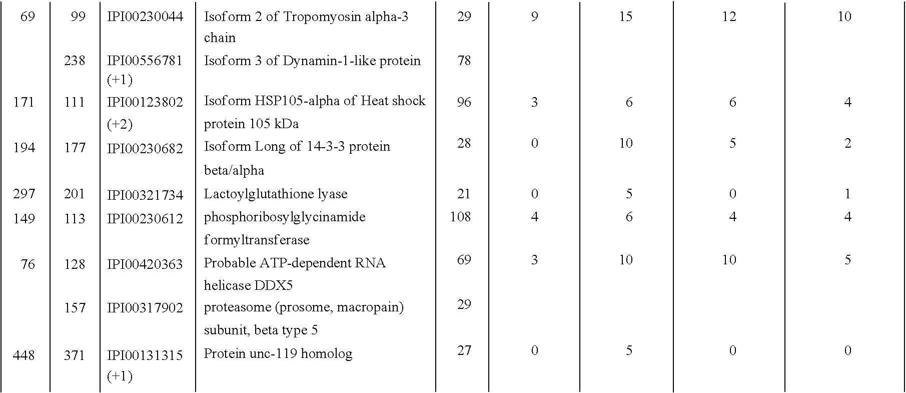

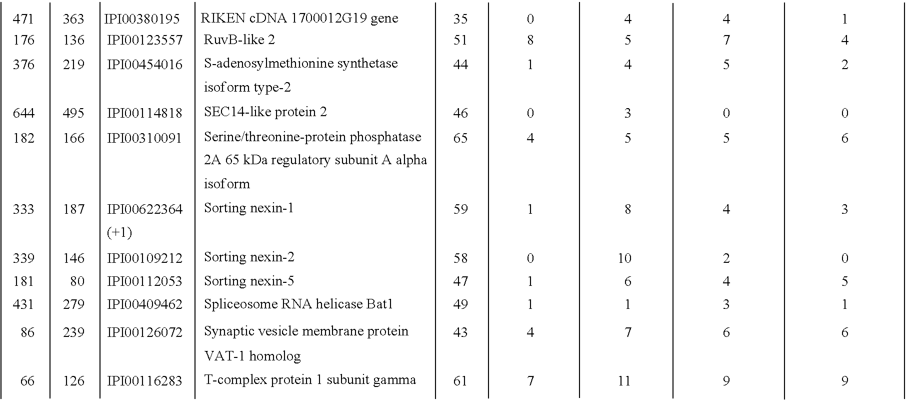

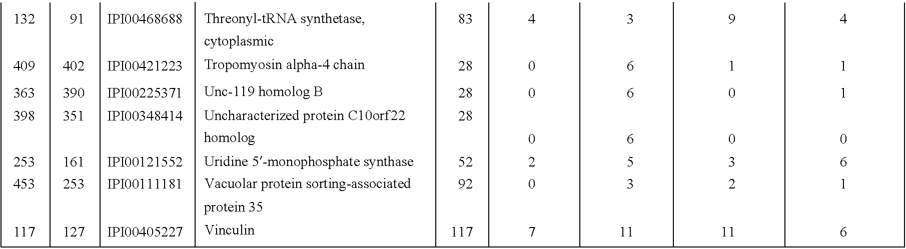

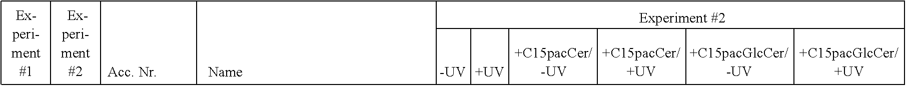

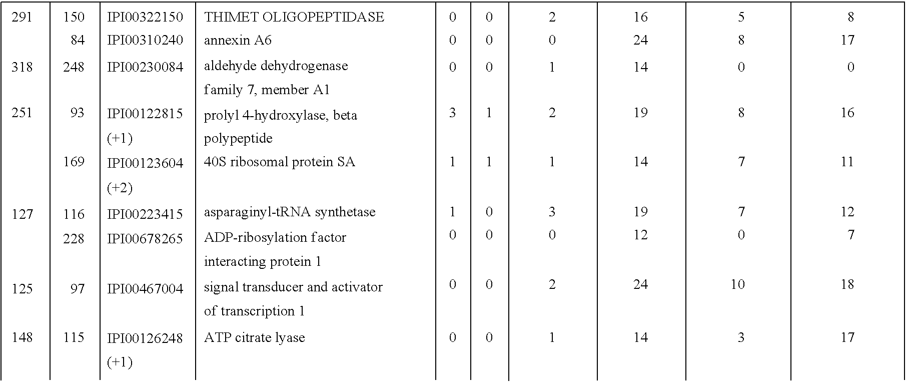

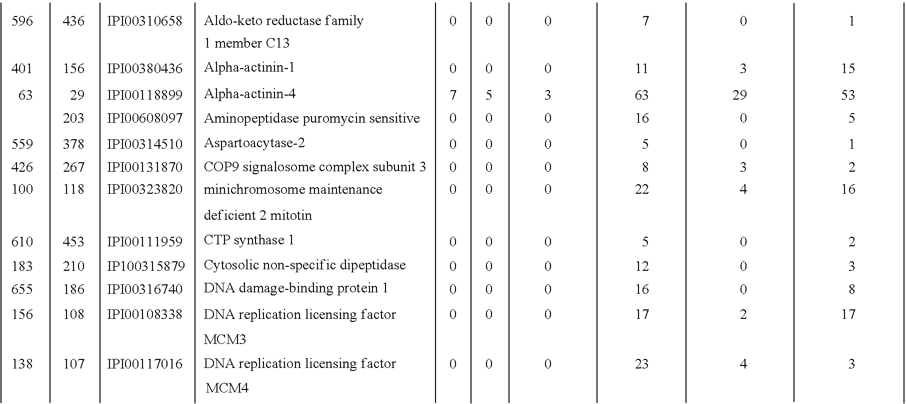

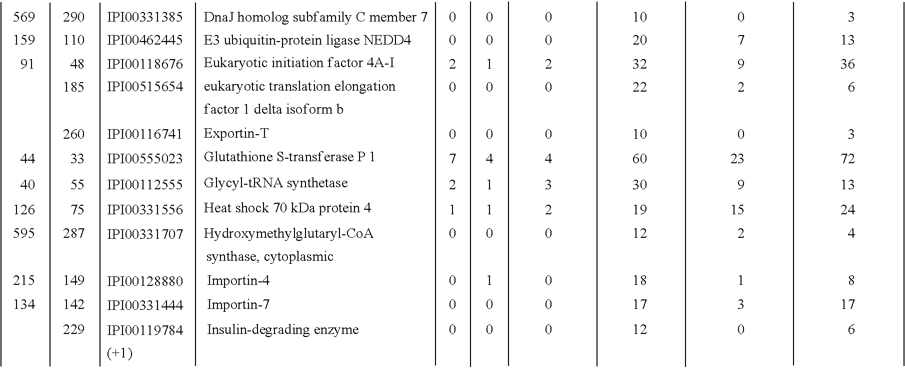

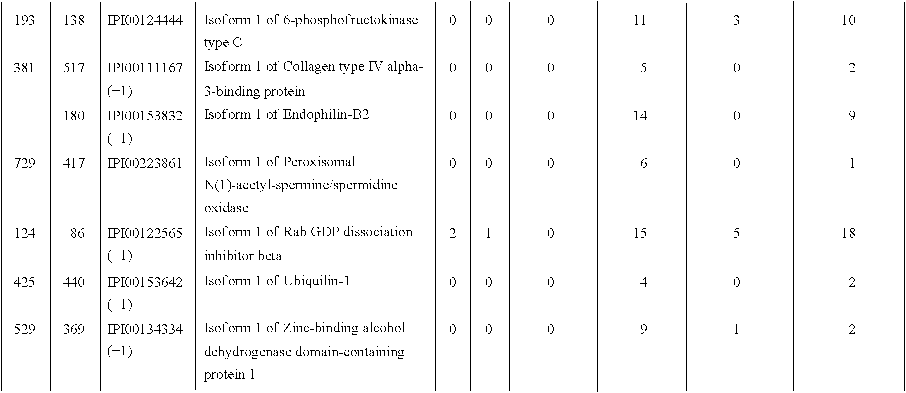

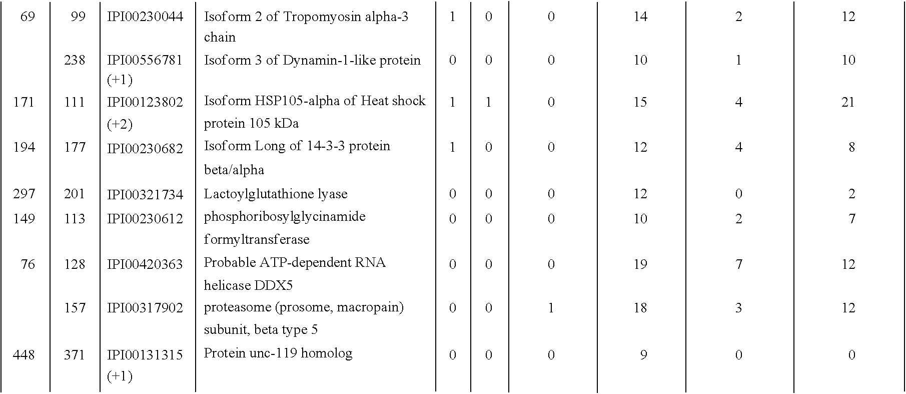

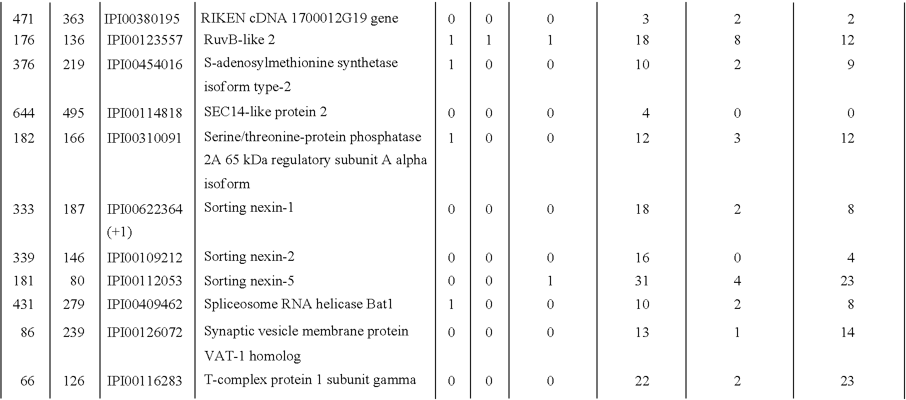

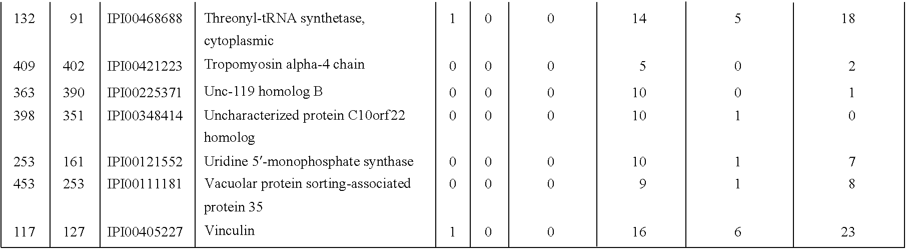

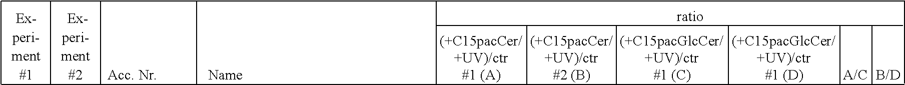

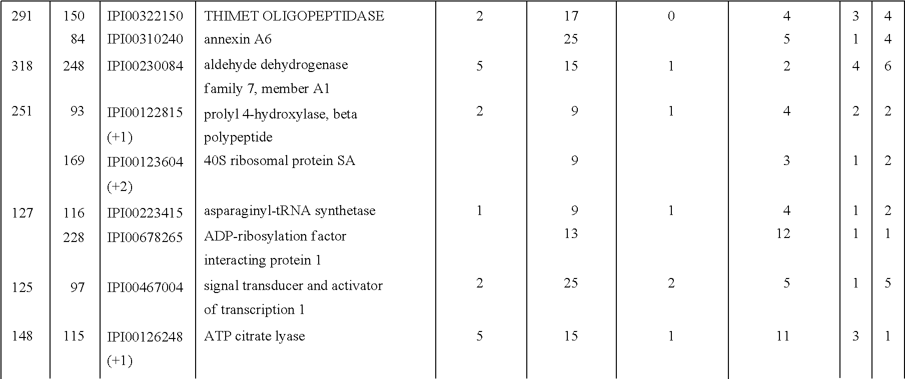

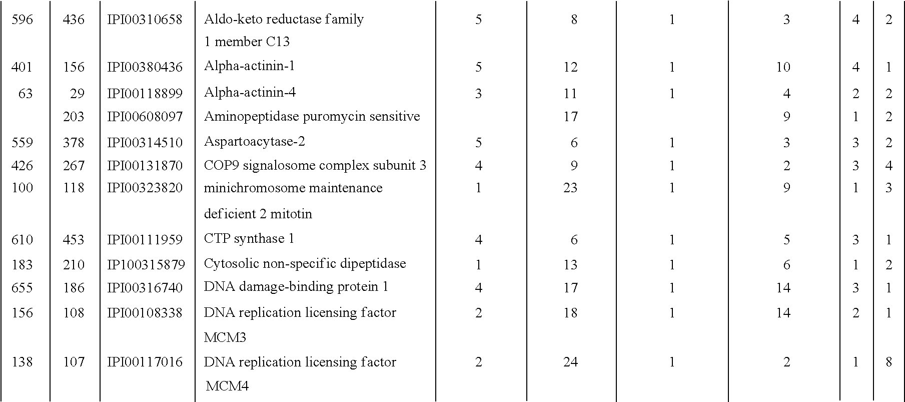

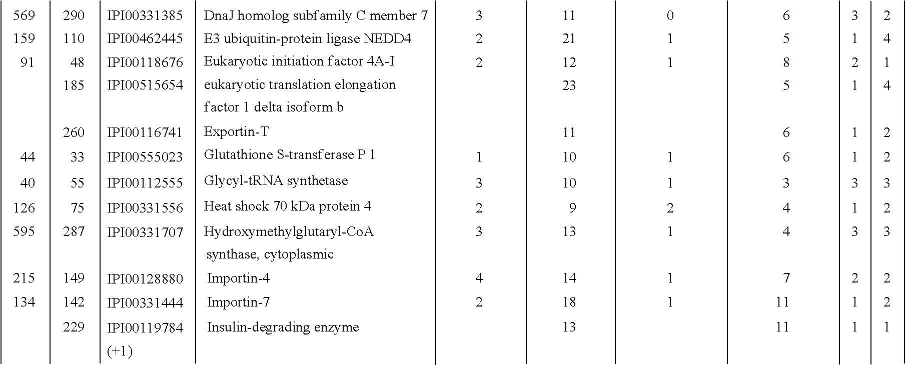

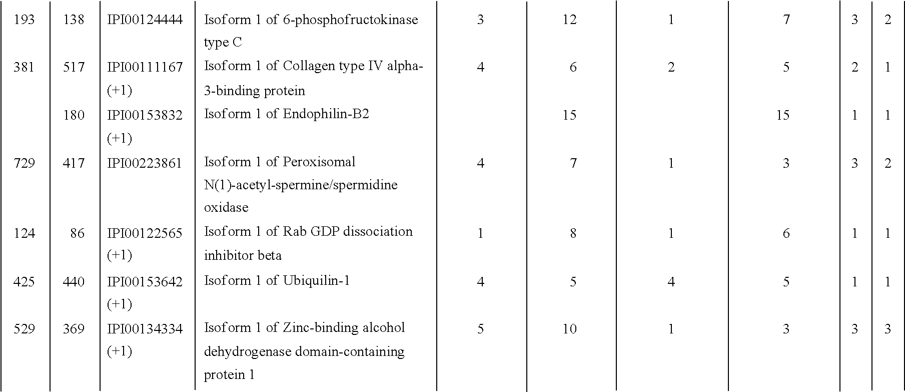

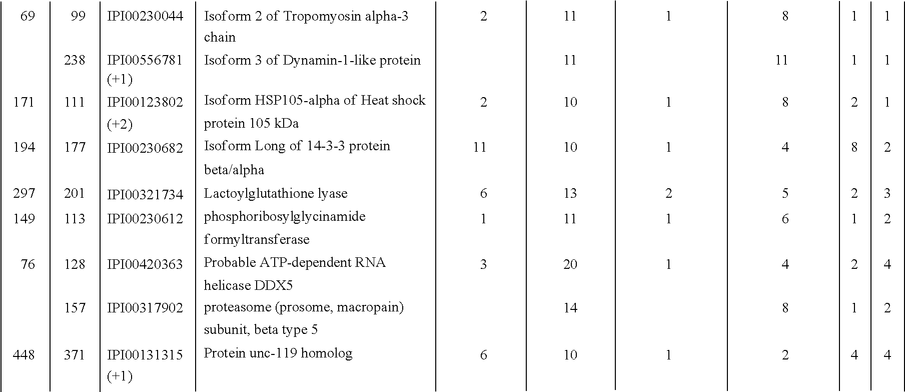

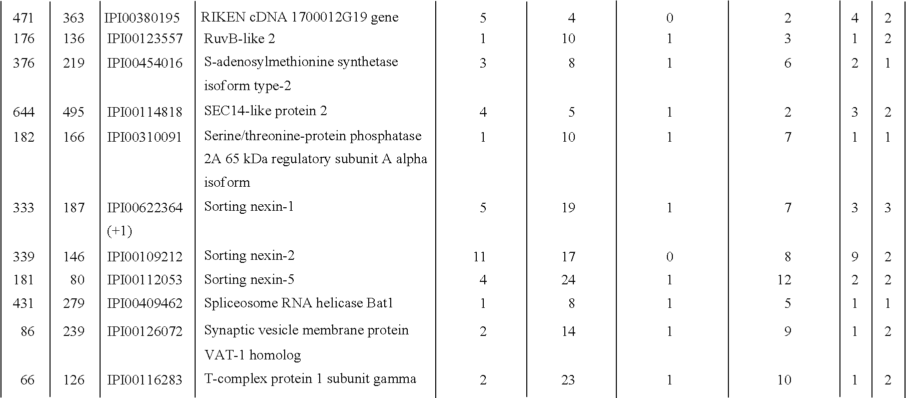

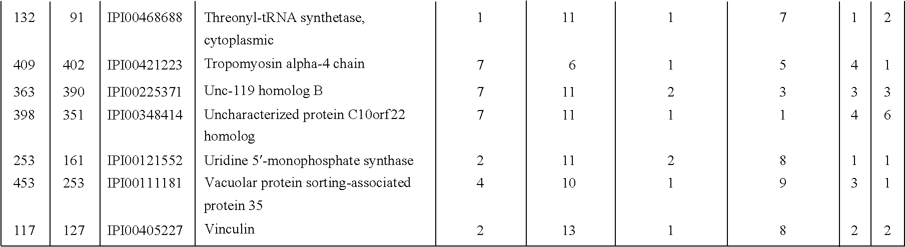

TABLE 5
A mass spectrometric analysis of two independent experiments
was performed and the subcellular localization of the identified
proteins (see Table 4) is shown.

| localization | Number of proteins |
|---|---|
| cytoplasm | 23 |
| nucleus | 8 |
| mitochondion | 7 |
| membrane | 5 |
| Golgi | 3 |
| cortical cytoskeleton | 3 |
| endoplasmic reticulum | 2 |
| podosome | 2 |
| stress fiber | 2 |
| peroxisome | 1 |
| small ribosomal subunit | 1 |
| signalosome | 1 |

TABLE 6
Given are numbers of proteins that represent a respective subset.

| | A | B |
|---|---|---|
| localization | | |
| cytoplasm | 51 | 14 |
| nucleus | 33 | 12 |
| endoplasmic reticulum | 23 | 46 |
| Golgi apparatus | 20 | 17 |
| cell membrane | 30 | 5 |
| mitochondrion | 31 | 30 |
| endosome | 9 | 4 |
| peroxisome | 9 | — |
| biological function | | |
| transport | 53 | 41 |
| lipid metabolism | 20 | 12 |
| signaling | 4 | 7 |
| Apoptosis | 7 | 5 |
| degradation | 2 | 4 |
| translocation | 5 | 3 |
| uncharacterized protein | 1 | 3 |
| unfolded protein response | 0 | 2 |
| ubl conjugation pathway | 0 | 2 |
| cell adhesion | 6 | 1 |

(A) Cluster of lipid-modified proteins that were exclusively found upon labeling with C15pacFA (lane III, FIG. 2, Panel D).
(B) Clusters of crosslinked proteins (proteins exclusively detected in lane IV, FIG. 2, Panel D).

What is claimed is:

1. A method for isolating or identifying a target protein interacting with a lipid, the method comprising:
a. providing a lipid precursor having a photoactivatable group and a terminal alkyne or azide group; or
a′. providing two lipid precursors wherein the first comprises a photoactivatable group and the second comprises a terminal alkyne or azide group;
b. contacting the lipid precursors according to a or a′ with cells and allowing the lipid precursor or precursors to be incorporated into lipids;
c. exposing said cells to photolysis wherein a target protein interacting with a lipid is covalently attached to said lipid having a terminal alkyne and/or an azide group; and
d. isolating or identifying the target protein by attaching a reporter molecule to the terminal alkyne or azide group.

2. The method according to claim 1, wherein the lipid precursor or precursors are contacted with cells in vivo.

3. The method according to claim 1, wherein the lipids are selected from the group consisting of phosphatidylcholine (PC), phosphatidylserine (PS), and phosphatidylethanolamine (PE).

4. The method according to claim 1, wherein the photoactivatable group is a diazirine group.

5. The method according to claim 1, wherein the reporter molecule comprises an alkyne or an azide group.

6. The method according to claim 1, wherein the protein is visualised by means of a fluorescent reporter molecule.

7. A method for isolating or identifying a target protein interacting with a lipid, the method comprising:
providing a lipid precursor having a photoactivatable group and a terminal alkyne or azide group;
contacting a cell with the lipid precursor and allowing the precursor to be incorporated into lipids by the cell;
exposing the cell to photolysis wherein a target protein interacting with a lipid having a terminal alkyne and/or an azide group is covalently attached to said lipid having a terminal alkyne and/or an azide group; and
isolating or identifying the target protein by attaching a reporter molecule to the terminal alkyne or azide group.

8. The method according to claim 7, wherein the lipid precursors us contacted with cells in vivo.

9. The method according to claim 7, wherein the lipids are selected from the group consisting of phosphatidylcholine (PC), phosphatidylserine (PS), and phosphatidylethanolamine (PE).

10. The method according to claim 7, wherein the photoactivatable group is a diazirine group.

11. The method according to claim 7, wherein the reporter molecule comprises an alkyne or an azide group.

12. The method according to claim 7, wherein the protein is visualised by means of a fluorescent reporter molecule.

13. A method for isolating or identifying a target protein interacting with a lipid, the method comprising:
providing two lipid precursors wherein the first lipid precursor comprises a photoactivatable group and the second lipid precursor comprises a terminal alkyne or azide group;
contacting a cell with the lipid precursors and allowing the precursors to be incorporated into lipids by the cell;
exposing the cell to photolysis wherein a target protein interacting with a lipid having a terminal alkyne and/or an azide group is covalently attached to said lipid having a terminal alkyne and/or an azide group; and
isolating or identifying the target protein by attaching a reporter molecule to the terminal alkyne or azide group.

14. The method according to claim 13, wherein the lipid precursors are contacted with cells in vivo.

15. The method according to claim 13, wherein the lipids are selected from the group consisting of phosphatidylcholine (PC), phosphatidylserine (PS), and phosphatidylethanolamine (PE).

16. The method according to claim 13, wherein the photoactivatable group is a diazirine group.

17. The method according to claim 13, wherein the reporter molecule comprises an alkyne or an azide group.

18. The method according to claim 13, wherein the protein is visualised by means of a fluorescent reporter molecule.
